(19) United States
(12) Patent Application Publication
(10) Pub. No.: US 2024/0069040 A1
(43) Pub. Date: Feb. 29, 2024

(54) DETECTION OF A RELAPSE IN A MULTIPLE SCLEROSIS PATIENT

(71) Applicants: Tianrong Yeo, Oxford (GB); OXFORD UNIVERSITY INNOVATION LIMITED, Oxford (GB)

(72) Inventors: Tianrong Yeo, Oxford (GB); Fay Probert, Oxford (GB); Daniel Anthony, Oxford (GB)

(73) Assignee: OXFORD UNIVERSITY INNOVATION LIMITED, Oxford (GB)

(21) Appl. No.: 18/043,181

(22) PCT Filed: Aug. 27, 2021
(86) PCT No.: PCT/GB2021/052234

§ 371 Date: Feb. 27, 2023

(30) Foreign Application Priority Data
Aug. 28, 2020 (GB) 2013554.7

(51) Int. Cl. G01N 33/68; G01N 33/92

(52) U.S. Cl. CPC G01N 33/6896; G01N 33/6812; G01N 33/92; G01N 2800/285; G01N 2800/52; G01N 2800/54

(57) ABSTRACT

The present invention is directed to methods for confirming that a multiple sclerosis (MS) patient is suffering from a relapse. In particular, methods comprising: comparing a concentration of one or more metabolite(s) present in a sample obtained from the patient with the concentration of the same one or more metabolite(s) in a reference standard, wherein the one or more metabolite(s) are selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, a lipoprotein having a βCH2 group, and an N-acetylated glycoprotein; and confirming or not that the patient is suffering from a relapse.

The present invention relates to multiple sclerosis and methods for confirming/predicting relapses and treating the same.

Multiple sclerosis (MS) is the most common cause of progressive disability in the Western world. MS can be divided into several subtypes: Relapsing-Remitting MS (RRMS), Secondary-Progressive MS (SPMS), and Primary-Progressive MS (PPMS). RRMS is defined by discrete and temporary periods of disability worsening/disease flare-up (relapses) followed by recovery or periods with no disability worsening or disease activity (remission). RRMS is the most common type of MS, affecting ˜85% of MS patients. The majority of RRMS patients will eventually proceed to develop SPMS. SPMS diagnosis, by definition, must follow an RRMS diagnosis. This type of MS is characterised by continued accrual of disability and progressive worsening of symptoms over time, typically with no more discrete relapses. PPMS is the rarest type of MS, affecting ˜10% of patients. In PPMS, the patient does not have a relapsing/remitting phase and enters the progressive phase from onset.

The vast majority (e.g. at least 80-85%) of MS patients experience clinical relapses during the course of their disease. From a clinical perspective, relapses represent an important component in the management of MS with regards to: (1) MS diagnosis (CIS vs. CDMS), (2) classification of clinical course (RRMS vs. PPMS during initial diagnosis, and RRMS vs. SPMS in the transitional phase), and (3) therapeutics decision-making (intensive treatments for ‘highly-active’ MS vs. less intensive treatments for ‘inactive’ MS). From a patient perspective, relapses represent a worsening of existing function with the uncertainty of incomplete recovery resulting in residual disability. It has been shown that there is association of relapses with disability worsening in the short-term, and there is evidence to suggest that high relapse rates early in the disease are predictive of long-term disability.

Within MS clinical research, relapse-related measures are still one of the primary endpoints in recent major phase III clinical trials for candidate therapies. Given that the current treatment paradigm is still largely guided by the presence of relapses, accurate determination of relapses is hugely important, especially when it is known that pseudo-relapses, which are transient neurophysiological disturbances due to infection, fever, heat-exposure or exercise, can cloud the clinical picture.

To date, the patient's history and neurological examination, as compared to previous examinations, are the principal methods used to establish relapses. This is often supplemented by MRI whereby the presence of GAD-enhancing or new/enlarging T₂ lesion/s within a CNS locale consistent with the neurological deficits offers unequivocal support for a clinical relapse. However, in clinical practice, a clinico-radiological discordance at time of relapse (although this paradox can exist at any point of the MS disease course) is often encountered. The presence of new clinical deficits without MRI correlates could be due to the poor sensitivity of conventional MRI to detect small lesions, particularly in the spinal cord, cortical grey matter and optic nerve. With regards to the use of biofluid markers to identify relapses, none have been validated (e.g. for widespread clinical use as confirmatory biomarkers for a relapse). Furthermore, there is no suitable prognostic method for predicting whether a patient will suffer a relapse.

There is thus a need for an improved method of confirming that an MS patient is suffering a relapse, and an improved method for determining prognosis of relapse.

The present invention provides a solution to at least one of the problems described above.

The present inventors have surprisingly found that a method comprising measuring a concentration of one or more metabolites described herein in a sample from a subject (patient) allows for an improved method for confirming that an MS patient is suffering a relapse. In other words, the inventors have demonstrated the utility for discriminatory metabolites in distinguishing patients ‘in relapse’ versus patients who are ‘inactive’ (e.g. in remission) or merely undergoing ‘pseudo-relapse’. Advantageously, even where the one or more metabolites described herein are present at a higher concentration in an MS relative to a healthy patient, said one or more metabolite is found at yet higher concentration an MS patient suffering a relapse (relative to an MS patient that is not suffering a relapse, e.g. is in remission).

The methods of the invention allow for improved confirmation that an MS patient is suffering a relapse per se, as well as determining prognosis of relapse (e.g. predicting an upcoming relapse in a patient currently in remission). Given the association of metabolites with disease state (e.g. relapse), methods of the invention allow for monitoring of a patient's response to therapy by measuring a concentration of one or more metabolites following administration of the therapy.

Advantageously, the methods of the invention are particularly accurate and/or sensitive and/or specific.

In a broad aspect, the invention provides a method for confirming that a multiple sclerosis (MS) patient is suffering from a relapse, the method comprising:
a. comparing a concentration of one or more metabolite(s) present in a sample obtained from the patient with the concentration of the same one or more metabolite(s) in a reference standard, wherein the one or more metabolite(s) are selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, and an N-acetylated glycoprotein; and
b. confirming that the patient is suffering from a relapse or not confirming the patient is suffering a relapse when based on the comparison.

Another broad aspect provides a method for confirming that a multiple sclerosis (MS) patient is suffering from a relapse, the method comprising:
a. comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
b. confirming that the patient is suffering from a relapse or not confirming the patient is suffering a relapse when based on the comparison.

One aspect of the invention provides a method for confirming that a multiple sclerosis (MS) patient is suffering from a relapse, the method comprising:
a. comparing a concentration of one or more metabolite(s) present in a sample obtained from the patient with the concentration of the same one or more metabolite(s) in a reference standard, wherein the one or more metabolite(s) are selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, and an N-acetylated glycoprotein; and
b. confirming that the patient is suffering from a relapse when:
   i. the concentration of one or more metabolite(s) selected from: lysine, asparagine, glucose, and a lipoprotein having a βCH₂ group is higher in the sample relative to the reference standard, wherein the reference standard is a non-relapse reference standard; and/or
   ii. the concentration of one or more metabolite(s) selected from: leucine, phenylalanine, p-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard; and/or
   iii. the concentration of one or more metabolite(s) selected from: lysine, asparagine, glucose, and a lipoprotein having a βCH₂ group is the same or higher in the sample relative to the reference standard, wherein the reference standard is a relapse reference standard; and/or
   iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard; or
c. not confirming that the patient is suffering from a relapse, or confirming that the patient is not suffering from a relapse, when:
   i. the concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
   ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard; and/or
   iii. the concentration of one or more metabolite(s) selected from: lysine, asparagine, glucose, and a lipoprotein having a βCH₂ group is lower in the sample relative to the reference standard, wherein the reference standard is a relapse reference standard; and/or
   iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

In another aspect of the invention, there is provided a method for confirming that a multiple sclerosis (MS) patient is suffering from a relapse, the method comprising:
a. comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
b. confirming that the patient is suffering from a relapse when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is lower in the sample relative to the reference standard, wherein the reference standard is a non-relapse reference standard; and/or
   iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard; and/or
   iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard; or
c. not confirming that the patient is suffering from a relapse, or confirming that the patient is not suffering from a relapse, when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard; and/or
   iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard; and/or
   iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a relapse reference standard.

Methods of the invention may find utility in diagnosing a patient as a (e.g. bone fide) MS patient, for example as opposed to a clinically isolated syndrome (CIS) patient that has not (or not yet) ‘converted’ to a clinically definite multiple sclerosis (CDMS).

Thus, in one embodiment, a method of the invention may comprise determining conversion of a subject from clinically isolated syndrome (CIS) to clinically definite multiple sclerosis (CDMS). In one embodiment, where a relapse is confirmed, the presence of a relapse may confirm that the patient has converted from CIS to CDMS. In such embodiments, the patient may be a patient that has previously been diagnosed to have CIS, and may not (yet) have been diagnosed to have MS, more preferably CDMS.

A further advantage of the invention includes the ability to classify the clinical course of MS in the patient via relapse monitoring. For example, relapsing-remitting MS (RRMS) and primary progressive MS (PPMS) may be distinguished during initial diagnosis, and RRMS vs. secondary progressive MS (SPMS) may be distinguished in the transitional phase.

Accurate relapse detection and monitoring, as achieved by the present invention, also plays an important role in therapeutics decision-making. For example, intensive treatments for ‘highly-active’ MS may be employed where regular relapses are detected/confirmed by a method described herein, or less intensive treatments may be employed for ‘inactive’ MS (e.g. where less regular or no relapses are detected/confirmed by a method described herein).

It should also be noted that within MS clinical research, relapse-related measures are still one of the primary endpoints in recent major phase III randomized controlled trials for candidate therapies. Thus, this invention finds utility when investigating a candidate therapy (e.g. within a clinical trial) as an objective measure of relapse.

Advantageously, the inventors have demonstrated that the one or more metabolite(s) described herein find utility in predicting relapse, e.g. an upcoming relapse in a patient that is currently in remission.

Another broad aspect of the invention provides a method for determining prognosis of a relapse in a multiple sclerosis (MS) patient (preferably an MS patient that is in remission), the method comprising:
a. comparing a concentration of one or more metabolite(s) present in a sample obtained from the patient with the concentration of the same one or more metabolite(s) in a reference standard, wherein the one or more metabolite(s) are selected from: leucine, lysine, asparagine, phenylalanine, glucose, β-hydroxybutyrate, myo-inositol, a lipoprotein having a —CH3 group of an HDL and/or LDL, a lipoprotein having a —CH3 group of a VLDL, a lipoprotein having —(CH2)n group of an HDL and/or LDL, a lipoprotein having a βCH2 group, and an N-acetylated glycoprotein (NAC); and
b. determining that the patient's prognosis is poor or not determining that the patient's prognosis is poor based on the comparison.

Another broad aspect of the invention provides a method for determining prognosis of a relapse in a multiple sclerosis (MS) patient (preferably an MS patient that is in remission), the method comprising:
a. comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
b. determining that the patient's prognosis is poor or not determining that the patient's prognosis is poor based on the comparison.

In another aspect of the invention, there is provided a method for determining prognosis of a relapse in a multiple sclerosis (MS) patient (preferably wherein the MS patient is in remission), the method comprising:
a. comparing a concentration of one or more metabolite(s) present in a sample obtained from the patient with the concentration of the same one or more metabolite(s) in a reference standard, wherein the one or more metabolite(s) are selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, and an N-acetylated glycoprotein; and
b. determining that the subject's prognosis is poor when:
   i. the concentration of one or more metabolite(s) selected from: lysine, asparagine, glucose, and a lipoprotein having a βCH₂ group is higher in the sample relative to the reference standard, wherein the reference standard is a non-relapse reference standard; and/or
   ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard; and/or
   iii. the concentration of one or more metabolite(s) selected from: lysine, asparagine, glucose, and a lipoprotein having a βCH₂ group is the same or higher in the sample relative to the reference standard, wherein the reference standard is a relapse reference standard; and/or
   iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard; or
c. not determining that the subject's prognosis is poor, or determining that the patient's prognosis is good, when:
   i. the concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
   ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard; and/or
   iii. the concentration of one or more metabolite(s) selected from: lysine, asparagine, glucose, and a lipoprotein having a βCH₂ group is lower in the sample relative to the reference standard, wherein the reference standard is a relapse reference standard; and/or
   iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

A yet further aspect of the invention provides a method for determining prognosis of a relapse in a multiple sclerosis (MS) patient (preferably wherein the MS patient is in remission), the method comprising:
a. comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
b. determining the patient's prognosis is poor when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is lower in the sample relative to the reference standard, wherein the reference standard is a non-relapse reference standard;
   iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard; and/or
   iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard; or
c. not determining that the patient's prognosis is poor, or determining that the patient's prognosis is good, when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard; and/or
   iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard; and/or
   iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a relapse reference standard.

In methods described herein for determining prognosis of a relapse in a MS patient, the sample may be a sample isolated from the patient ≤30 days prior to onset of relapse; preferably ≤25 days prior to onset of relapse; more preferably ≤20 days prior to onset of relapse. Preferably, in methods described herein for determining prognosis of a relapse in MS patient, the sample may be a sample isolated from the patient ≤15 days (more preferably prior ≤14 days) prior to onset of relapse.

Another broad aspect of the invention provides a method for monitoring a multiple sclerosis (MS) patient's response to therapy, wherein the patient is suffering or suspected of suffering from a relapse, or is at risk of suffering a relapse, the method comprising:
a. providing a sample obtained from the patient, wherein the patient has received therapy for a relapse;
b. comparing a concentration of one or more metabolite(s) present in a sample obtained from the patient with the concentration of the same one or more metabolite(s) in a reference standard (preferably wherein the reference standard is a reference standard that is representative of the concentration of said one or more metabolite(s) in a sample obtained from the subject pre-administration of the therapy), wherein the one or more metabolite(s) are selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, and an N-acetylated glycoprotein; and
c. determining that the patient is responsive to the therapy or not determining that the patient is responsive to the therapy based on the comparison.

Another broad aspect of the invention provides a method for monitoring a multiple sclerosis (MS) patient's response to therapy, wherein the patient is suffering or suspected of suffering from a relapse, or is at risk of suffering a relapse, the method comprising:
a. providing a sample obtained from the patient, wherein the patient has received therapy for a relapse;
b. comparing an intensity of one or more chemical shift region(s) of a ¹H-NMR spectrum of a sample obtained from a subject with the intensity of the same one or more chemical shift region(s) of a ¹H-NMR reference standard (preferably wherein the reference standard is a reference standard that is representative of the intensity of said one or more chemical shift region(s) in a sample obtained from the subject pre-administration of the therapy), wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
c. determining that the patient is responsive to the therapy or not determining that the patient is responsive to the therapy based on the comparison.

One aspect of the invention provides a method for monitoring a multiple sclerosis (MS) patient's response to therapy, wherein the patient is suffering or suspected of suffering from a relapse, or is at risk of suffering a relapse, the method comprising:
a. providing a sample obtained from the patient, wherein the patient has received therapy for a relapse;
b. comparing a concentration of one or more metabolite(s) present in the sample obtained from the patient with the concentration of the same one or more metabolite(s) in a reference standard, wherein the one or more metabolite(s) are selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, a lipoprotein having a βCH₂ group, and an N-acetylated glycoprotein; and
c. determining that the patient is responsive to the therapy when:
   i. the concentration of one or more metabolite(s) selected from: lysine, asparagine, glucose, and a lipoprotein having a βCH₂ group is lower in the sample relative to the reference standard, wherein the reference standard is a reference standard that is representative of the concentration of said one or more metabolite(s) in a sample obtained from the subject pre-administration of the therapy; and/or
   ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is higher in the sample relative to the reference standard, wherein the reference standard is a reference standard that is representative of the concentration of said one or more metabolite(s) in a sample obtained from the subject pre-administration of the therapy; or
d. not determining that the patient is responsive to the therapy, or determining that the patient is not responsive to the therapy, when:
   i. the concentration of one or more metabolite(s) selected from: lysine, asparagine, glucose, and a lipoprotein having a βCH₂ group is the same or higher in the sample relative to the reference standard, wherein the reference standard is a reference standard that is representative of the concentration of said one or more metabolite(s) in a sample obtained from the subject pre-administration of the therapy; and/or
   ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is the same or lower in the sample relative to the reference standard, wherein the reference standard is a reference standard that is representative of the concentration of said one or more metabolite(s) in a sample obtained from the subject pre-administration of the therapy.

One aspect of the invention provides a method for monitoring a multiple sclerosis (MS) patient's response to therapy, wherein the patient is suffering or suspected of suffering from a relapse, or is at risk of suffering a relapse, the method comprising:
a. providing a sample obtained from the patient, wherein the patient has received therapy for a relapse;
b. comparing an intensity of one or more chemical shift region(s) of a ¹H-NMR spectrum of the sample obtained from the subject with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
c. determining that the patient is responsive to the therapy when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a reference standard that is representative of the intensity of said one or more chemical shift region(s) in a sample obtained from the subject pre-administration of the therapy; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a reference standard that is representative of the intensity of said one or more chemical shift region(s) in a sample obtained from the subject pre-administration of the therapy; or
d. not determining that the patient is responsive to the therapy, or determining that the patient is not responsive to the therapy, when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a reference standard that is representative of the intensity of said one or more chemical shift region(s) in a sample obtained from the subject pre-administration of the therapy; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a reference standard that is representative of the intensity of said one or more chemical shift region(s) in a sample obtained from the subject pre-administration of the therapy.

Another aspect of the invention provides a method for monitoring a multiple sclerosis (MS) patient's response to therapy, wherein the patient is suffering or suspected of suffering from a relapse, or is at risk of suffering a relapse, the method comprising:
a. providing a sample obtained from the patient, wherein the patient has received therapy for a relapse;
b. comparing a concentration of one or more metabolite(s) present in a sample obtained from the patient with the concentration of the same one or more metabolite(s) in a reference standard, wherein the one or more metabolite(s) are selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, and an N-acetylated glycoprotein; and
c. determining that the patient is responsive to the therapy when:
   i. the concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
   ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard; and/or
   iii. the concentration of one or more metabolite(s) selected from: lysine, asparagine, glucose, and a lipoprotein having a βCH₂ group is the lower in the sample relative to the reference standard, wherein the reference standard is a relapse reference standard; and/or
   iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the higher in the sample relative to the reference standard, wherein the reference standard is a relapse reference standard; or
d. not determining that the patient is responsive to the therapy, or determining that the patient is not responsive to the therapy, when:
   i. the concentration of one or more metabolite(s) selected from: lysine, asparagine, glucose, and a lipoprotein having a βCH₂ group is higher in the sample relative to the reference standard, wherein the reference standard is a non-relapse reference standard; and/or
   ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard; and/or
   iii. the concentration of one or more metabolite(s) selected from: lysine, asparagine, glucose, and a lipoprotein having a βCH₂ group is the same or higher in the sample relative to the reference standard, wherein the reference standard is a relapse reference standard; and/or
   iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard.

Another aspect of the invention provides a method for monitoring a multiple sclerosis (MS) patient's response to therapy, wherein the patient is suffering or suspected of suffering from a relapse, or is at risk of suffering a relapse, the method comprising:
a. comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
b. determining that the patient is responsive to the therapy when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard; and/or
   iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard; and/or
   iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the higher in the sample relative to the reference standard, wherein the reference standard is a relapse reference standard; or
c. not determining that the patient is responsive to the therapy, or determining that the patient is not responsive to the therapy, when:
   i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard; and/or
   ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the lower in the sample relative to the reference standard, wherein the reference standard is a non-relapse reference standard; and/or
   iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard; and/or
   iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard.

In all aspects related to a “method for monitoring a MS patient's response to therapy response to therapy” described herein, the “therapy” is preferably a drug; more preferably a candidate drug. Said drug (for example, candidate drug) may be comprised within a pharmaceutical composition, optionally together with one or more pharmaceutically acceptable excipient(s).

In one aspect the invention provides a method, the method comprising:
a. obtaining a biofluid sample derived from a patient, optionally a multiple sclerosis patient suffering, or suspected of suffering, from a relapse;
b. assaying the biofluid sample for a concentration of one or more metabolite(s) in the biofluid sample selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, and an N-acetylated glycoprotein; optionally:
c. comparing the assayed concentration with the concentration of the same one or metabolite(s) in a reference standard; and
d. confirming that the patient is suffering from a relapse when:
   i. the assayed concentration of one or more metabolite(s) selected from: lysine, asparagine, glucose, and a lipoprotein having a βCH₂ group is higher in the sample relative to the reference standard, wherein the reference standard is a non-relapse reference standard; and/or
   ii. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard; and/or
   iii. the assayed concentration of one or more metabolite(s) selected from: lysine, asparagine, glucose, and a lipoprotein having a βCH₂ group is the same or higher in the sample relative to the reference standard, wherein the reference standard is a relapse reference standard; and/or
   iv. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard; or
e. not confirming that the patient is suffering from a relapse, or confirming that the patient is not suffering from a relapse, when:
   i. the assayed concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
   ii. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard; and/or
   iii. the assayed concentration of one or more metabolite(s) selected from: lysine, asparagine, glucose, and a lipoprotein having a βCH₂ group is lower in the sample relative to the reference standard, wherein the reference standard is a relapse reference standard; and/or
   iv. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

In one aspect, the invention provides a method, the method comprising:
a. providing a sample obtained from a patient, optionally wherein the patient is a multiple sclerosis (MS) patient having, or suspected having, a relapse; and
b. assaying the sample for an intensity of one or more chemical shift region(s) of a ¹H-NMR spectrum, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; optionally
c. comparing the assayed intensity with the intensity of the same one or more chemical shift region(s) of a ¹H-NMR spectrum in a reference standard; and
d. confirming that the patient is suffering from a relapse when:
   i. the assayed intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard; and/or
   ii. the assayed intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is lower in the sample relative to the reference standard, wherein the reference standard is a non-relapse reference standard; and/or
   iii. the assayed intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard; and/or
   iv. the assayed intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard; or
e. not confirming that the patient is suffering from a relapse, or confirming that the patient is not suffering from a relapse, when:
   i. the assayed intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard; and/or
   ii. the assayed intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard; and/or
   iii. the assayed intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard; and/or
   iv. the assayed intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a relapse reference standard.

Another aspect of the invention provides a method for predicting whether a multiple sclerosis patient will suffer a relapse, the method comprising:
a. obtaining a biofluid sample derived from a multiple sclerosis patient, preferably an MS patient that is in remission;
b. assaying the biofluid sample for a concentration of one or more metabolite(s) in the biofluid sample selected from leucine, lysine, asparagine, phenylalanine, glucose, β-hydroxybutyrate, myo-inositol, a lipoprotein having a —CH3 group of an HDL and/or LDL, a lipoprotein having a —CH3 group of a VLDL, a lipoprotein having —(CH2)n group of an HDL and/or LDL, a lipoprotein having a βCH₂ group, and an N-acetylated glycoprotein;
c. comparing the assayed concentration with the concentration of the same one or more metabolite(s) in a reference standard; and
d. determining that the patient will suffer a relapse based on the comparison when:
   i. the assayed concentration of one or more metabolite(s) selected from: lysine, asparagine, glucose, and a lipoprotein having a βCH₂ group is higher in the sample relative to the reference standard, wherein the reference standard is a non-relapse reference standard; and/or
   ii. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard; and/or
   iii. the assayed concentration of one or more metabolite(s) selected from: lysine, asparagine, glucose, and a lipoprotein having a βCH₂ group is the same or higher in the sample relative to the reference standard, wherein the reference standard is a relapse reference standard; and/or
   iv. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard; or
e. not determining that the patient will suffer a relapse based on the comparison, or determining that the subject will not suffer a relapse based on the comparison, when:
   i. the assayed concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
   ii. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard; and/or
   iii. the assayed concentration of one or more metabolite(s) selected from: lysine, asparagine, glucose, and a lipoprotein having a βCH₂ group is lower in the sample relative to the reference standard, wherein the reference standard is a relapse reference standard; and/or
   iv. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

In one aspect, the invention provides a method for predicting prognosis of a relapse in an MS patient, the method comprising:
a. obtaining a biofluid sample derived from a MS patient;
b. assaying the biofluid sample for a concentration of: one or more metabolite(s) in the biofluid sample selected from leucine, lysine, asparagine, phenylalanine, glucose, β-hydroxybutyrate, myo-inositol, a lipoprotein having a —CH3 group of an HDL and/or LDL, a lipoprotein having a —CH3 group of a VLDL, a lipoprotein having —(CH2)n group of an HDL and/or LDL, a lipoprotein having a βCH2 group, and an N-acetylated glycoprotein; optionally
c. comparing the assayed concentration with the concentration of the same one or more metabolite(s) in a reference standard; and
d. determining that the subject's prognosis is poor based on the comparison when:
   i. the assayed concentration of one or more metabolite(s) selected from: lysine, asparagine, glucose, and a lipoprotein having a βCH₂ group is higher in the sample relative to the reference standard, wherein the reference standard is a non-relapse reference standard; and/or
   ii. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard; and/or
   iii. the assayed concentration of one or more metabolite(s) selected from: lysine, asparagine, glucose, and a lipoprotein having a βCH₂ group is the same or higher in the sample relative to the reference standard, wherein the reference standard is a relapse reference standard; and/or
   iv. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard; or
e. not determining that the subject's prognosis is poor based on the comparison, or determining the patient's prognosis is good based on the comparison, when:
   i. the assayed concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
   ii. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard; and/or
   iii. the assayed concentration of one or more metabolite(s) selected from: lysine, asparagine, glucose, and a lipoprotein having a βCH₂ group is lower in the sample relative to the reference standard, wherein the reference standard is a relapse reference standard; and/or
   iv. the assayed concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

A method of the invention may further comprise: comparing a concentration of isoleucine and/or serum neurofilament light chain (NfL) present in a sample obtained from a subject with the concentration of isoleucine and/or NfL, respectively, in a reference standard; and
a. (i) confirming that the patient is suffering from a relapse, or (ii) determining the patient's prognosis is poor, when:
   i. the concentration of NfL is higher in the sample relative to the reference standard, wherein the reference standard is a non-relapse reference standard; and/or
   ii. the concentration of isoleucine is lower in the sample relative to the reference standard, wherein the reference standard is a non-relapse reference standard; and/or
   iii. the concentration of NfL is the same or higher in the sample relative to the reference standard, wherein the reference standard is a relapse reference standard; and/or
   iv. the concentration of isoleucine is the same or lower in the sample relative to the reference standard, wherein the reference standard is a relapse reference standard; or
b. (i) not confirming that the patient is suffering from a relapse, or (ii) not determining the patient's prognosis is poor, when:
   i. the concentration of NfL is the same or lower in the sample relative to the reference standard, wherein the reference standard is a non-relapse reference standard; and/or
   ii. the concentration of isoleucine is the same or higher in the sample relative to the reference standard, wherein the reference standard is a non-relapse reference standard; and/or
   iii. the concentration of NfL is lower in the sample relative to the reference standard, wherein the reference standard is a relapse reference standard; and/or
   iv. the concentration of isoleucine is higher in the sample relative to the reference standard, wherein the reference standard is a relapse reference standard.

A method of the invention may further comprise: comparing a concentration of isoleucine and/or serum neurofilament light chain (NfL) present in a sample obtained from a subject with the concentration of isoleucine and/or NfL, respectively, in a reference standard; and
a. determining that the patient is responsive to the therapy when:
   i. the concentration of NfL is the same or lower in the sample relative to the reference standard, wherein the reference standard is a non-relapse reference standard; and/or
   ii. the concentration of isoleucine is the same or higher in the sample relative to the reference standard, wherein the reference standard is a non-relapse reference standard; and/or
   iii. the concentration of NfL is lower in the sample relative to the reference standard, wherein the reference standard is a relapse reference standard; and/or
   iv. the concentration of isoleucine is higher in the sample relative to the reference standard, wherein the reference standard is a relapse reference standard; or
b. not determining that the patient is responsive to the therapy when:
   i. the concentration of NfL is the higher in the sample relative to the reference standard, wherein the reference standard is a non-relapse reference standard; and/or
   ii. the concentration of isoleucine is lower in the sample relative to the reference standard, wherein the reference standard is a non-relapse reference standard; and/or
   iii. the concentration of NfL is the same or higher in the sample relative to the reference standard, wherein the reference standard is a relapse reference standard; and/or
   iv. the concentration of isoleucine is the same or lower in the sample relative to the reference standard, wherein the reference standard is a relapse reference standard.

A method of the invention may further comprise comparing an intensity of a chemical shift region of a ¹H-NMR spectrum of a sample obtained from a subject with the intensity of the same one or more chemical shift region of a ¹H-NMR reference standard, wherein the chemical shift region is 0.92-0.97 ppm, 1.00-1.03 ppm, 1.22-1.28 ppm, 1.43-1.51 ppm, 1.94-2.01 ppm, and/or 3.65-3.68 ppm; and
a. (i) confirming that the patient is suffering from a relapse, or (ii) determining the patient's prognosis is poor, when:
   i. the intensity of said chemical shift region of the ¹H-NMR spectrum is lower in the sample relative to the reference standard, wherein the reference standard is a non-relapse reference standard; and/or
   ii. the intensity of said chemical shift region of the ¹H-NMR spectrum is the same or lower in the sample relative to the reference standard, wherein the reference standard is a relapse reference standard; or
b. (i) not confirming that the patient is suffering from a relapse, or (ii) not determining the patient's prognosis is poor, when:
   i. the intensity of said chemical shift region of the ¹H-NMR spectrum is the same or higher in the sample relative to the reference standard, wherein the reference standard is a non-relapse reference standard; and/or
   ii. the intensity of said chemical shift region of the ¹H-NMR spectrum is higher in the sample relative to the reference standard, wherein the reference standard is a relapse reference standard.

A method of the invention (e.g. a method for monitoring a multiple sclerosis (MS) patient's response to therapy, wherein the patient is suffering or suspected of suffering from a relapse, or is at risk of suffering a relapse) may further comprise:
a. comparing a concentration of isoleucine and/or serum neurofilament light chain (NfL) present in a sample obtained from a subject with the concentration of isoleucine and/or NfL, respectively, in a reference standard; and
b. determining that the patient is responsive to the therapy when:
   i. the concentration of NfL is lower in the sample relative to the reference standard, wherein the reference standard is a reference standard that is representative of the concentration of said one or more metabolite(s) in a sample obtained from the subject pre-administration of the therapy; and/or
   ii. the concentration of isoleucine is higher in the sample relative to the reference standard, wherein the reference standard is a reference standard that is representative of the concentration of said one or more metabolite(s) in a sample obtained from the subject pre-administration of the therapy; or
c. not determining that the patient is responsive to the therapy when:
   i. the concentration of NfL is the same or higher in the sample relative to the reference standard, wherein the reference standard is a reference standard that is representative of the concentration of said one or more metabolite(s) in a sample obtained from the subject pre-administration of the therapy; and/or
   ii. the concentration of isoleucine is the same or lower in the sample relative to the reference standard, wherein the reference standard is a reference standard that is representative of the concentration of said one or more metabolite(s) in a sample obtained from the subject pre-administration of the therapy.

A method of the invention (e.g. a method for monitoring a multiple sclerosis (MS) patient's response to therapy, wherein the patient is suffering or suspected of suffering from a relapse, or is at risk of suffering a relapse) may further comprise:
a. comparing an intensity of a chemical shift region of a ¹H-NMR spectrum of a sample obtained from a subject with the intensity of the same one or more chemical shift region of a ¹H-NMR reference standard, wherein the chemical shift region is 0.92-0.97 ppm, 1.00-1.03 ppm, 1.22-1.28 ppm, 1.43-1.51 ppm, 1.94-2.01 ppm, and/or 3.65-3.68 ppm; and
b. determining that the patient is responsive to the therapy when:
   i. the intensity of said chemical shift region of the ¹H-NMR spectrum is higher in the sample relative to the reference standard, wherein the reference standard is a reference standard that is representative of the intensity of said one or more chemical shift region(s) in a sample obtained from the subject pre-administration of the therapy; or
c. not determining that the patient is responsive to the therapy when:
   ii. the intensity of said chemical shift region of the 1H-NMR spectrum is the same or lower in the sample relative to the reference standard, wherein the reference standard is a reference standard that is representative of the intensity of said one or more chemical shift region(s) in a sample obtained from the subject pre-administration of the therapy.

Reference to a “multiple sclerosis patient” may embrace a patient having one of the four basic MS disease types (also called types or courses or phenotypes), for example as have been defined by the International Advisory Committee on Clinical Trials of MS in 2013: clinically isolated syndrome (CIS), relapsing remitting (RRMS), secondary progressive (SPMS) and primary progressive (PPMS). For example, multiple sclerosis patient may have CIS (in such cases, the patient may have been diagnosed to have CIS, but not yet diagnosed to have CDMS). The multiple sclerosis patient may have SPMS. The multiple sclerosis patient may have PPMS.

In a preferable embodiment, the multiple sclerosis patient has relapsing remitting multiple sclerosis (RRMS). In other words, the patient may be an RRMS patient.

A “relapse” of MS (also known as an exacerbation, attack or flare-up) means an episode of the occurrence of new symptoms or the worsening of old symptoms. A relapse can be (very) mild, or severe enough to interfere with a person's ability to function. Symptoms vary from person to person and from one exacerbation to another. For example, the exacerbation might include an episode of optic neuritis (e.g. caused by inflammation of the optic nerve that impairs vision), or problems with balance or severe fatigue. Some relapses produce only one symptom (e.g. related to inflammation in a single area of the central nervous system). Other relapses may cause two or more symptoms at the same time (e.g. related to inflammation in more than one area of the central nervous system). The “relapse” may be referred to as an “acute relapse”.

Suitably, to be a true exacerbation, the attack lasts at least 24 hours and can be separated from a (the) previous attack by at least 30 days. Suitably, the relapse occurs in the absence of infection, or other cause of symptoms. Most exacerbations last from a few days to several weeks or even months. Relapses often occur without warning, but are sometimes associated with a period of illness or stress. The symptoms of a relapse may disappear altogether, with or without treatment, although some symptoms often persist, with repeated attacks happening over several years.

Periods between attacks are known as periods of “remission”. These can last for months or even years at a time. An MS patient that is in remission is in a disease phase defined by mild or no symptoms of MS, and the absence of an (e.g. acute) relapse.

Thus, methods of the invention preferably allow to distinguish between an MS patient “in relapse” versus an MS patient “in remission”. Additionally or alternatively, methods of the invention may allow to distinguish between an MS patient “in relapse” versus an MS patient having a “pseudo-relapse”. The term “pseudo-relapse” is described elsewhere herein.

A method of the invention may further comprise identifying the presence or absence or a symptom of relapse in the patient, and confirming that the patient is suffering a relapse when a symptom is present, or not confirming that the subject is suffering a relapse when a symptom is absent. Examples of such symptoms include: fatigue, difficulty walking, vision problems (such as blurred vision), problems controlling the bladder, numbness (or tingling) in different parts of the body, muscle stiffness and spasms, problems with balance and co-ordination, and/or problems with thinking, learning and planning. Symptoms may also include optic neuritis (e.g. caused by inflammation of the optic nerve that impairs vision), or problems with balance or severe fatigue, and/or inflammation in one or more area of the central nervous system.

A method of the invention may comprise a step of measuring a concentration of one or more metabolite(s) present in a sample obtained from a subject.

A method of the invention may comprise a step of obtaining a ¹H-NMR spectrum of a sample obtained from a subject.

The concentrations of the metabolites in a sample can be measured using any suitable technique known in the art. By way of example, the following techniques may be used alone or in combination to detect and quantify molecules in solution, and are thus suitable for determining metabolite concentrations: Nuclear Magnetic Resonance (NMR) spectroscopy, mass spectrometry, gas chromatography, ultraviolet (UV) spectrometry (for example in combination with high-performance liquid chromatography [HPLC] as HPLC-UV), infrared spectroscopy, and a biochemical assay. A metabolite is preferably identified using NMR, more preferably ¹H-NMR. The biochemical assay may be an enzymatic assay.

In one embodiment, the concentration of one or more metabolites is determined using NMR spectroscopy. In one embodiment, the concentration of one or more metabolites is determined using mass spectrometry. In one embodiment, the concentration of one or more metabolites is determined using HPLC-UV. In one embodiment, the concentration of one or more metabolites is determined using infrared spectroscopy.

The concentration of a metabolite in a sample can be expressed in a number of different ways, for example as a molar concentration (number of moles of metabolite per unit volume of sample) or a mass concentration (mass of metabolite per unit volume of sample). Alternatively, the concentration of a metabolite can be expressed as parts per million (ppm) or parts per billion (ppb). Such ways of expressing the concentration of a molecule in solution are known in the art. In some embodiments, a concentration of a metabolite may be expressed relative to a standard or to another metabolite within the sample. For example, when techniques such as NMR are employed a concentration may be expressed as a relative spectral intensity.

Thus, in one embodiment, the concentration of a metabolite in a sample is the molar concentration of said metabolite. In one embodiment, the concentration of a metabolite in a sample is the mass concentration of said metabolite.

The concentration of a metabolite in a sample may be expressed in absolute terms, for example as an absolute molar concentration or absolute mass concentration. Alternatively, the concentration of a metabolite in a sample can be expressed by comparison to the concentration of a different metabolite in the same sample (i.e. in relative terms). By way of example, the concentration of a metabolite in the sample can be normalised by comparison to the concentration of a different reference metabolite within the same sample.

The methods described herein are particularly sensitive and allow for accurate and/or sensitive and/or specific determination and/or diagnosis when using only one metabolite. Notably, even where the concentration of a metabolite has not been found to be statistically-significantly changed when compared to a reference standard, said metabolite has utility in a method of the invention, especially where used in combination with a further metabolite and/or when compared to multiple reference standards.

In one embodiment, a metabolite for use in the invention is a lipoprotein. A lipoprotein may be a very low density lipoprotein (VLDL), a low density lipoprotein (LDL) or a high density lipoprotein (HDL). In some embodiments, the methods employs the use of at least two of: a VLDL, a LDL, and an HDL.

A lipoprotein may be detected, and/or its concentration measured, by detecting a chemical group of the lipoprotein, for example a —CH₃ group of a lipoprotein. When using NMR, certain chemical shift ranges are characteristic of such groups of the various density lipoproteins, as described below.

In one embodiment, a method of the invention utilises a —(CH₂)ₙ group of an HDL and/or LDL. A ¹H-NMR chemical shift range of 1.15-1.30 ppm may be characteristic of a —(CH₂)ₙ group of an HDL and/or LDL. Thus, methods of the invention may comprise comparison of the concentration of a lipoprotein having a —(CH₂)ₙ group of an HDL and/or LDL with the concentration of said metabolite in a reference standard. Said lipoprotein may be referred to as “lipoprotein (CH₂)ₙ (HDL/LDL dominated)”.

In one embodiment, a method of the invention utilises a —CH₃ group of an HDL and/or LDL. A ¹H-NMR chemical shift range of 0.80-0.86 ppm may be characteristic of a —CH₃ group of an HDL and/or LDL. Thus, methods of the invention may comprise comparison of the concentration of a lipoprotein having —CH₃ group of an HDL and/or LDL with the concentration of said metabolite in a reference standard. Said lipoprotein may be referred to as “lipoprotein CH3 (HDL/LDL dominated)”.

In one embodiment, a method of the invention utilises a —CH₃ group of a VLDL. A ¹H-NMR chemical shift range of 0.86-0.92 ppm may be characteristic of a —CH₃ group of a VLDL. Thus, methods of the invention may comprise comparison of the concentration of a lipoprotein having a —CH₃ group of a VLDL with the concentration of said metabolite in a reference standard. Said lipoprotein may be referred to as “lipoprotein CH₃ (VLDL dominated)”.

In one embodiment, a method utilises a —(CH₂)ₙ group of a VLDL. A ¹H-NMR chemical shift range of 1.30-1.39 ppm may be characteristic of a —(CH₂)ₙ group of a VLDL. Thus, methods of the invention may comprise comparison of the concentration of a lipoprotein having a —CH₃ group of a VLDL with the concentration of said metabolite in a reference standard. Said lipoprotein may be referred to as “lipoprotein —(CH₂)ₙ (VLDL dominated)”.

In one embodiment, a method utilises a βCH₂ group of a lipoprotein. A ¹H-NMR chemical shift range of 1.53-1.61 ppm may be characteristic of a βCH₂ group of a lipoprotein. Thus, methods of the invention may comprise comparison of the concentration of a lipoprotein having a βCH₂ group with the concentration of said metabolite in a reference standard. Said lipoprotein may be referred to as “a lipoprotein having a βCH₂”.

In one embodiment, a method utilises an —N(CH₃)₃ group of a lipoprotein. A ¹H-NMR chemical shift range of 3.17-3.31 ppm may be characteristic of an —N(CH₃)₃ group of a lipoprotein. Thus, methods of the invention may comprise comparison of the concentration of a lipoprotein having an —N(CH₃)₃ group with the concentration of said metabolite in a reference standard. Said lipoprotein may be referred to as “a lipoprotein having an —N(CH₃)₃”.

In one embodiment, a metabolite for use in the invention is an N-acetylated glycoprotein (NAC). Said metabolite may be defined via a ¹H-NMR chemical shift range of 1.93-2.10 ppm. Said metabolite may be referred to as “NAC₁/═CH—CH₂—CH₂—”.

In one embodiment, a metabolite for use in the invention is lysine. Said metabolite may be defined via one or more ¹H-NMR chemical shift range(s) of 1.37-1.55 ppm, 1.65-1.75 ppm, 1.83-1.94 ppm, and/or 3.00-3.05 ppm. Preferably, said metabolite may be defined via ¹H-NMR chemical shift ranges of 1.37-1.55 ppm, 1.65-1.75 ppm, 1.83-1.94 ppm, and 3.00-3.05 ppm.

In one embodiment, a metabolite for use in the invention is glucose. Said metabolite may be defined via one or more ¹H-NMR chemical shift range(s) of 3.17-3.95 ppm, 4.63-4.66 ppm, and/or 5.22-5.25 ppm. Preferably, said metabolite may be defined via ¹H-NMR chemical shift ranges of 3.17-3.95 ppm, 4.63-4.66 ppm, and 5.22-5.25 ppm.

In one embodiment, a metabolite for use in the invention is β-hydroxybutyrate and/or β-hydroxybutyric acid. Said metabolites may be defined via one or more ¹H-NMR chemical shift range(s) of 1.19-1.21 ppm and/or 2.27-2.45 ppm. Preferably, said metabolites may be defined via ¹H-NMR chemical shift ranges of 1.19-1.21 ppm and 2.27-2.45 ppm.

In one embodiment, a metabolite for use in the invention is β-hydroxybutyrate. Said metabolite may be defined via one or more ¹H-NMR chemical shift range(s) of 1.19-1.21 ppm and/or 2.27-2.45 ppm. Preferably, said metabolite may be defined via ¹H-NMR chemical shift ranges of 1.19-1.21 ppm and 2.27-2.45 ppm.

In one embodiment, a metabolite for use in the invention is myo-inositol. Said metabolite may be defined via one or more ¹H-NMR chemical shift range(s) of 3.63-3.65 ppm, 3.53-3.58 ppm, 3.93-3.98 ppm, and/or 3.25-3.29 ppm. Preferably, said metabolite may be defined via ¹H-NMR chemical shift ranges of 3.63-3.65 ppm, 3.53-3.58 ppm, 3.93-3.98 ppm, and 3.25-3.29 ppm.

In one embodiment, a metabolite for use in the invention is leucine. Said metabolite may be defined via one or more ¹H-NMR chemical shift range(s) of 0.94-0.98 ppm, 1.62-1.78 ppm, and/or 3.70-3.79 ppm. Preferably, said metabolite may be defined via ¹H-NMR chemical shift ranges of 0.94-0.98 ppm, 1.62-1.78 ppm, and 3.70-3.79 ppm.

In one embodiment, a metabolite for use in the invention is isoleucine. Said metabolite may be defined via one or more ¹H-NMR chemical shift range(s) of 0.92-0.97 ppm, 1.00-1.03 ppm, 1.22-1.28 ppm, 1.43-1.51 ppm, 1.94-2.01 ppm, and/or 3.65-3.68 ppm. Preferably, said metabolite may be defined via ¹H-NMR chemical shift ranges of 0.92-0.97 ppm, 1.00-1.03 ppm, 1.22-1.28 ppm, 1.43-1.51 ppm, 1.94-2.01 ppm, and 3.65-3.68 ppm.

In one embodiment, a metabolite for use in the invention is asparagine. Said metabolite may be defined via one or more ¹H-NMR chemical shift range(s) of 2.80-3.00 ppm, and/or 3.96-4.02. Preferably, said metabolite may be defined via ¹H-NMR chemical shift ranges of 2.8-3.00 ppm, and 3.96-4.02.

In one embodiment, a metabolite for use in the invention is phenylalanine. Said metabolite may be defined via one or more ¹H-NMR chemical shift range(s) of 7.32-7.44 ppm, 3.1-3.3 ppm, and/or 3.9-4.0 ppm. Preferably, said metabolite may be defined via ¹H-NMR chemical shift ranges of 7.32-7.44 ppm, 3.1-3.3 ppm, and 3.9-4.0 ppm.

In one aspect, the metabolites herein may instead be referred to by their ¹H-NMR chemical shift range(s), as described above.

In some embodiments, more than one metabolite may be employed, i.e. a plurality of metabolites may be employed. In a preferred embodiment, at least 2 metabolites are employed in a method described herein.

The term “one or more” when used in the context of a metabolite described herein may mean at least 2, 3, 4, 5, 6, 7, 8, 9, 10, 11, 12, 13 or 14 metabolites. For example, the term “one or more” when used in the context of a metabolite described herein may mean at least 2, 3, 4, 5, 6, 7, 8, 9, 10, 12, or 13 metabolites. Suitably, the term “one or more” when used in the context of a metabolite described herein may mean at least 2, 3, 4, 5, 6, 7, 8, 9, 10, 11 or 12 metabolites. When carrying out a method herein, it is preferred that those metabolites that are highest ranked in Table 3 are used, for example, where 2 metabolites are used, it is preferred that these are the 2 highest ranking metabolites. It is preferred that at least two metabolites selected from lysine, asparagine, isoleucine and leucine (more preferably at least two metabolites selected from lysine, asparagine, and leucine) are employed.

In one embodiment, the invention utilizes two or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes two or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes two or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein. Preferably, two or more metabolites selected from: lysine, asparagine, isoleucine and leucine; more preferably, two or more metabolites selected from lysine, asparagine, and leucine.

In one embodiment, the invention utilizes three or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes three or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes three or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein. Preferably, three or more metabolites selected from: lysine, asparagine, isoleucine and leucine; more preferably, three or more metabolites selected from lysine, asparagine, and leucine.

In one embodiment, the invention utilizes four or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes four or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes four or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein. Preferably, four or more metabolites selected from: lysine, asparagine, isoleucine and leucine.

In one embodiment, the invention utilizes five or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes five or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes five or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes six or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes six or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes six or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes seven or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes seven or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes seven or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes eight or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes eight or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes eight or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes nine or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes nine or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes nine or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes ten or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes ten or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes ten or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes eleven or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes eleven or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes eleven or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes twelve or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes twelve or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes twelve or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes thirteen or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes thirteen or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes thirteen or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein.

In one embodiment, the invention utilizes fourteen or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes fourteen or more metabolites selected from: lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, NfL and isoleucine. In one embodiment, the invention utilizes fourteen or more metabolites selected from: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, and an N-acetylated glycoprotein.

In one embodiment, the invention utilizes all of the following metabolites: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH₂ group, an N-acetylated glycoprotein, NfL and isoleucine. In one embodiment, the invention utilizes all of the following metabolites: leucine, lysine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein. In one embodiment, the invention utilizes all of the following metabolites: lysine, asparagine, leucine, isoleucine. In one embodiment, the invention utilizes all of the following metabolites: lysine, asparagine, leucine.

In one embodiment, the invention utilizes one or more selected from leucine, lysine, asparagine, and isoleucine; and one or more selected from phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, and NfL (preferably and one or more selected from phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein).

In one embodiment, the invention utilizes two or more (preferably three or more) selected from leucine, lysine, asparagine, and isoleucine; and one or more selected from phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, an N-acetylated glycoprotein, and NfL (preferably and one or more selected from phenylalanine, glucose, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, a lipoprotein having a βCH2 group, and an N-acetylated glycoprotein).

In a preferred embodiment, the invention utilizes all of the following metabolites: lysine, asparagine, leucine, isoleucine and NfL. In a particularly preferred embodiment, the invention utilizes all of the following metabolites: lysine, asparagine, leucine, isoleucine.

Methods of the present invention are also based on the identification of chemical shift regions of ¹H-NMR spectra that allow for accurate and/or sensitive and/or specific diagnosis of cancer, a primary cancer, and/or a secondary cancer. By way of background, carrying out ¹H-NMR produces a spectrum, as is known in the art. The spectrum can be characterised according to the chemical shift positions (in ppm), which define peak positions, and the intensity of the peaks. Intensity (i.e. peak/spectral intensity) corresponds to the concentration of a chemical (e.g. a metabolite) present in a sample. Intensity may be determined by any method known in the art, such as determining an area under the peak. The Examples herein define a particularly preferred method for carrying out ¹H-NMR to produce a spectrum for use in the present invention.

The chemical shifts quoted herein may be considered to encompass a value that deviates from the quoted value by ±0.01 ppm, preferably a value that deviates from the quoted value by less than ±0.01 ppm, more preferably by 0 ppm.

In one embodiment, a suitable volume of a sample is diluted with an appropriate buffer (preferably having a pH meter reading of 7.4) and solvent. Preferably, the sample comprises D₂O. Preferably, a suitable volume (e.g. 150 μl) of a sample is diluted with (e.g. 450 μl) sodium phosphate buffer prepared in D₂O (pH meter reading of 7.4).

Said samples may be processed to remove any precipitate prior to carrying out NMR.

Preferably, the chemical shift regions quoted herein are reported relative to lactate —CH₃ referenced at 1.33 ppm.

In one embodiment, the ¹H-NMR is carried out on samples at 298K. Preferably, the ¹H-NMR is carried out on samples at 310K.

Most preferably, the chemical shift regions herein have been defined by carrying out “the ¹H-NMR assay” described herein. “The ¹H-NMR assay” comprises the following steps:
(a) diluting 150 μL of sample with 450 μL of 75 mM sodium phosphate buffer prepared in D₂O (pH meter reading of 7.4);
(b) centrifuging said diluted sample at 16,000×g for 3 minutes to remove any precipitate;
(c) transferring the supernatant to a 5 mm NMR tube;
(d) obtaining an NMR spectrum of the sample, wherein the spectrum is obtained using a 1D NOESY presaturation scheme for attenuation of the water resonance with a 2 s presaturation using a 700-MHz NMR spectrometer equipped with a helium-cooled cryogenic probe (e.g. a 700-MHz Bruker AVIII spectrometer operating at 16.4T equipped with a ¹H (¹³C/¹⁵N) TCI cryoprobe) at a sample temperature of 298K or 310K (preferably 310K);
(e) zero-filling resulting free induction decays (FIDs) by a factor of 2 and multiplying by an exponential function corresponding to 0.30 Hz line broadening prior to Fourier transformation;
(f) phasing and baseline correcting (using a 3^{rd} degree polynomial) the spectrum; and
(g) referencing chemical shifts to the lactate-CH₃ doublet resonance at 5=1.33 ppm; and
(h) optionally, when the sample is a serum or plasma sample, in addition to the 1D NOESY presaturation scheme, a spin-echo Carr-Purcell-Meiboom-Gill (CPMG) sequence with a T interval of 400 μs, 80 loops, 32 data collections, an acquisition time of 1.5 s, and a relaxation delay of 2 s may be used to supress broad signals arising from large molecular weight blood components; and
(i) optionally, for blood plasma and blood serum spectra, the regions between 0.20-4.70 ppm and 5.00-9.60 ppm may be divided in to 0.01 ppm width ‘buckets’. For urine spectra, the regions between 0.20-4.70 ppm and 5.00-5.70 and 5.96-9.60 ppm may be divided in to 0.01 ppm width ‘buckets’; and
(j) optionally the absolute value of the integral of each spectral bucket may be Pareto scaled; and
(k) optionally, resonances may be assigned by reference to literature values [Anal Biochem 325:260-272, J Pharm Biomed Anal 33:1103-1115] and the Human Metabolome Database [Nucleic Acids Res 41:D801-807. doi:10.1093/nar/gks1065, Nucleic Acids Res 37:D603-610. doi:10.1093/nar/gkn810, Nucleic Acids Res 35:D521-526. doi:10.1093/nar/gkl923] and further confirmed by inspection of the 2D spectra, spiking of known compounds, and 1 D-TOCSY spectra.

In one embodiment, the invention encompasses ¹H-NMR techniques carried out under conditions other than those defined herein, for example via the AXINON® lipoFIT® system (numares).

In the unlikely event that said techniques lead to one or more different chemical shift region(s), said different chemical shift region(s) are encompassed by the present invention so long as the different chemical shift region(s) correspond to the chemical shift region(s) presented herein when carried out using “the ¹H-NMR assay” described herein.

A method of the invention may utilize one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. A method of the invention may utilize one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. A method of the invention may utilize one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

The methods comprising the use of chemical shift regions of ¹H-NMR spectra are accurate and/or sensitive and/or specific when using only one chemical shift region. Notably, even where the intensity of a chemical shift region has not been found to be statistically-significantly changed when compared to a reference standard, said chemical shift region has utility in a method of the invention, especially where used in combination with a further chemical shift region and/or when compared to multiple reference standards.

In some embodiments, more than one chemical shift region may be employed, i.e. a plurality of chemical shift regions may be employed. In a preferred embodiment, at least 2 chemical shift regions are employed in a method described herein.

The term “one or more” when used in the context of a chemical shift region described herein may mean at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 32 chemical shift regions. For example, the term “one or more” when used in the context of a chemical shift region described herein may mean at least 2, 3, 4, 5, 6, 7, 8, 9, 10, 11, 12, 13, 14, 15, 16, 17, 18, 19, 20, 21, 22, 23, 24, 25, or 26 chemical shift regions. For example, the term “one or more” when used in the context of a metabolite described herein may mean at least 2, 3, 4, 5, 6, 7, 8, 9, 10, 11, 12, 13, 14, 15, 16, 17, 18, 19, 20, 21, 22, 23, 24, 25, or 26 metabolites. When carrying out a method herein, it is preferred that those chemical shift regions that are highest ranked in Table 3 are used, for example, where 2 chemical shift regions are used, it is preferred that these are the 2 highest ranking chemical shift regions. Preferably, at least two chemical shift regions employed are selected from 0.94-0.98 ppm, 1.62-1.78 ppm, 3.70-3.79 ppm, 1.37-1.55 ppm, 1.65-1.75 ppm, 1.83-1.94 ppm, 3.00-3.05 ppm, 2.80-3.00 ppm, 3.96-4.02 ppm, 0.92-0.97 ppm, 1.00-1.03 ppm, 1.22-1.28 ppm, 1.43-1.51 ppm, 1.94-2.01 ppm, and 3.65-3.68 ppm are employed.

Thus, in one embodiment, the invention utilizes two or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, two or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In one embodiment, the invention utilizes three or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, three or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In one embodiment, the invention utilizes four or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, four or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In one embodiment, the invention utilizes five or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, five or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In one embodiment, the invention utilizes ten or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, ten or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In one embodiment, the invention utilizes fifteen or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, fifteen or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In one embodiment, the invention utilizes twenty or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, twenty or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In one embodiment, the invention utilizes twenty-five or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. Preferably, twenty-five or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm.

The invention may utilize all of the following chemical shift region(s): 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.93-2.10 ppm, 0.92-0.97 ppm, 1.00-1.03 ppm, 1.22-1.28 ppm, 1.43-1.51 ppm, 1.94-2.01 ppm, and 3.65-3.68 ppm. In a particularly preferred embodiment, the invention utilizes all of the following chemical shift region(s): 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm.

In some embodiments, chemical shift ranges encompassing a plurality of the narrower ranges provided above are employed, for example one or more of the following chemical shift region(s) may be employed 0.80-0.92 ppm, 1.15-1.39 ppm, 1.53-1.61 ppm, 1.88-2.10 ppm, 2.20-2.49 ppm, 2.51-2.70 ppm, 3.17-3.95 ppm, 4.63-4.66 ppm, and 5.22-5.38 ppm.

The terms “subject” and “patient” are used synonymously herein. The “subject” (aka. patient) may be a mammal, and preferably the subject is a human subject. A subject may be a subject that has or, is suspected of having, multiple sclerosis. In some embodiments, e.g. when it is desired to confirm that the patient is suffering a relapse, a patient (e.g. subject) may be a MS patient that is suffering, or suspected of suffering, a relapse. The patient may be a MS patient that is at risk of suffering from a relapse.

Preferably, a patient is a patient that has presented with non-specific symptom(s)/sign(s) of a relapse (e.g. an MS relapse). A method of the invention may further comprise detecting the presence or the absence of such non-specific symptom(s)/sign(s), and confirming or not confirming (respectively) that the patient is suffering a relapse. Such non-specific symptoms/signs may include optic neuritis (e.g. caused by inflammation of the optic nerve that impairs vision), problems with balance or severe fatigue, unexplained inflammation in the central nervous system (CNS), fatigue, difficulty walking, vision problems (such as blurred vision), problems controlling the bladder, numbness (or tingling) in different parts of the body, muscle stiffness and spasms, problems with balance and co-ordination, and/or problems with thinking, learning and planning, and/or GP clinical suspicion of a relapse (GP ‘gut feeling’).

Confirmation of the presence or absence of relapse may also be corroborated by reference to magnetic resonance imaging (MRI) data obtained upon analysis of the patient. For example, such MRI data may confirm the presence or absence of GAD-enhancing or new/enlarging T₂ lesion/s within a CNS locale (e.g. consistent with the neurological deficits), offering further support for the presence or absence of a clinical relapse.

The sample that is to be tested using the method of the invention may be derived from any suitable biofluid. Thus, the sample is preferably a biofluid sample. In one embodiment, the biofluid is selected from blood, cerebrospinal fluid (CSF), or urine that has been obtained from a subject. Preferably the sample is a blood sample.

The term “blood” comprises whole blood, blood serum (henceforth “serum”) and blood plasma (henceforth “plasma”), preferably serum. Serum and plasma are derived from blood and thus may be considered as specific subtypes within the broader genus “blood”. Processes for obtaining serum or plasma from blood are known in the art. For example, it is known in the art that blood can be subjected to centrifugation in order to separate red blood cells, white blood cells, and plasma. Serum is defined as plasma that lacks clotting factors. Serum can be obtained by centrifugation of blood in which the clotting process has been triggered. Optionally, this can be carried out in specialised centrifuge tubes designed for this purpose.

A sample for use in a method of the present invention can be derived from a biofluid that has undergone processing after being obtained from a test subject. Alternatively, a sample can be derived from a biofluid that has not undergone any processing after being obtained from a test subject.

The methods of the invention may use samples that have undergone minimal or zero processing before testing. This provides a significant advantage over prior art methods in terms of time, cost and practicality. By way of example, a blood sample obtained from a test subject may be tested directly using the method of the present invention, without further processing. Serum and plasma samples can be readily obtained from blood samples using simple and readily available techniques that are well known in the art, as described above.

A sample for use in a method of the invention may be a cell-free sample. In other words, the sample of the invention may be processed to remove cells. The term “cell-free samples” are samples that contain substantially no cells. The term “substantially no” when used in the context of cells herein may mean less than 10,000, 5,000, 1,000, 100 or 10 cells/ml. The term “substantially no” when used in the context of cells herein preferably means less than 1,000 cells/ml, more preferably no cells. In some embodiments, the term “substantially no” when used in the context of cells herein may be expressed in absolute amounts. For example, the term “substantially no” when used in the context of cells herein may mean less than 10,000, 5,000, 1,000, 100 or 10 cells. Preferably less than 1,000 cells, more preferably no cells.

The methods of the invention comprise comparing a concentration of a metabolite to a reference standard. Similarly, the methods of the invention may comprise comparing an intensity of one or more chemical shift regions of a ¹H-NMR spectrum of a sample with a reference standard.

In one embodiment, a reference standard comprises (or consists of) a sample (e.g. a biofluid sample described herein) obtained from a reference subject or subjects (e.g. patient or patients), wherein the reference subject is a subject other than the subject being tested in a method of the invention.

In one embodiment, a “reference standard” comprises (or consists of) a set of data relating to the concentration of one or more metabolites, and/or the intensity of one or more chemical shift regions of a ¹H-NMR spectrum, obtained from a reference subject or subjects, wherein the reference subject is a subject other than the subject being tested in a method of the invention. The set of data may be derived by measuring the concentration of said one or more metabolites and/or measuring the intensity of one or more chemical shift regions of a ¹H-NMR spectrum. Said measuring may be carried out using any suitable technique known in the art or described herein. It is particularly preferred that the set of data corresponding to the reference sample are obtained (or have been obtained) using the same or a similar technique used to obtain the concentration of the one or more metabolites or one or more chemical shift regions (respectively) in the sample being tested. As part of his common general knowledge, the skilled person knows which variables in an experimental protocol can be varied without affecting comparability of data and those that cannot be varied, and will thus select an appropriate experimental protocol to ensure comparability between a sample from a subject and a reference standard. Most preferably, the same technique and protocol will be used to obtain the concentration of the one or more metabolites or one or more chemical shift regions (respectively) in the sample and in the reference standard.

In some embodiments, a reference standard may be a dataset constructed based on a knowledge of metabolite concentrations, and/or chemical shift intensities, that are indicative of the presence of a relapse, or the absence of a relapse. In some embodiments, a reference standard may be constructed based on metabolite concentrations and/or chemical shift intensities for a known population of MS patients suffering from a relapse and/or a known population of MS patients not suffering from a relapse (e.g. non-relapse population, also known as patients that are in remission). In other words, in some embodiments, a reference standard does not correspond to an actual sample obtained from a reference subject. However, it is preferred that a reference standard comprises (or consists of) a set of data relating to the concentration of one or more metabolites, and/or the intensity of one or more chemical shift regions of a ¹H-NMR spectrum, obtained from a reference subject or subjects, wherein the reference subject is a subject other than the subject being tested in a method of the invention.

In one embodiment, the reference standard comprises (or consists of) a set of data relating to the concentration of said one or more metabolites, and/or the intensity of one or more chemical shift regions of a ¹H-NMR spectrum, in a sample or samples derived from a single reference subject. In other embodiments, the reference standard comprises (or consists of) a set of data relating to the concentration of said one or more metabolites, and/or the intensity of one or more chemical shift regions of a ¹H-NMR spectrum, in a sample or samples derived from a plurality of reference subjects (e.g. two or more reference subjects). Thus, in one embodiment, the reference standard is derived by pooling data obtained from two or more (e.g. three, four, five, 10, 15, 20 or 25) reference subjects and calculating an average (for example, mean or median) concentration for each metabolite, and/or an average intensity of one or more chemical shift regions of a ¹H-NMR spectrum. Thus, the reference standard may reflect average concentrations of said one or more metabolites, and/or average intensities of chemical shift regions of a ¹H-NMR spectrum, in a given population of reference subjects. Said concentrations and/or intensities may be expressed in absolute or relative terms, in the same manner as described above in relation to the sample that is to be tested using the method of the invention.

In one embodiment, a method of the invention comprises the use of a plurality of reference standards. In such embodiments, a method may comprise the use of a non-relapse reference standard and a relapse reference standard. The use of multiple reference standards is particularly preferred when it is necessary to confirm whether or not a patient is suffering a relapse, but also whether a patient is predicted to suffer a relapse.

In some embodiments, the methods of the present invention comprise comparing measured concentrations of metabolites to the concentration of said metabolites (respectively) in both a relapse and a non-relapse reference standard (or a plurality of relapse and non-relapse reference standards) and determining to which reference standard the sample is most similar (thus allowing a determination/diagnosis according to a method of the invention).

In some embodiments, the methods of the present invention comprise comparing measured intensities of chemical shift regions of a ¹H-NMR spectrum to the intensity of said chemical shift regions of a ¹H-NMR spectrum (respectively) in both a relapse and a non-relapse reference standard (or a plurality of relapse and non-relapse reference standards) and determining to which reference standard the sample is most similar (thus allowing a determination/diagnosis according to a method of the invention).

A metabolite concentration in a reference standard may have been obtained (e.g. quantified) prior to carrying out a method of the invention.

When comparing concentrations between the sample and the reference standard, the way in which the concentrations are expressed is matched between the sample and the reference standard. Thus, an absolute concentration can be compared with an absolute concentration, and a relative concentration can be compared with a relative concentration.

An intensity of a chemical shift region of a ¹H-NMR spectrum in a reference standard may have been obtained (e.g. quantified) prior to carrying out a method of the invention.

When comparing intensities between the sample and the reference standard, the way in which the intensities are expressed is matched between the sample and the reference standard. Thus, an absolute intensity can be compared with an absolute intensity, and a relative intensity can be compared with a relative concentration. Moreover, the ¹H-NMR protocol used for obtaining a spectrum for the sample and reference standard should preferably be the same.

The reference standard is preferably derived from the same sample type (e.g. biofluid) as the sample that is being tested, thus allowing for an appropriate comparison between the metabolites and/or chemical shifts.

The methods of the present invention are in vitro methods. Thus, the methods can be carried out in vitro on an isolated sample that has been obtained from a subject.

The methods of the invention may comprise comparing the (measured) concentrations of one or more metabolites to make a diagnosis. Thus, said (measured) concentrations may correlate with the presence of a relapse. Said diagnosis may be based on measuring/identifying a concentration difference. The term “concentration difference” embraces both positive and negative differences. Thus, a concentration difference can mean that the concentration of a metabolite is higher in the sample being tested than in the reference standard. Alternatively, a concentration difference can mean that the concentration of a metabolite is lower in the sample than in the reference standard.

Similarly, methods of the invention may comprise comparing the (measured) intensities of one or more chemical shift regions of a ¹H-NMR spectrum to make a diagnosis. Thus, said (measured) intensities may correlate with the presence of a relapse. Said diagnosis may be based on measuring/identifying a difference in intensity. The term “difference in intensity” embraces both positive and negative differences. Thus, a difference in intensity can mean that the intensity of a chemical shift region is higher in the sample being tested than in the reference standard. Alternatively, a difference in intensity can mean that the intensity of a chemical shift region is lower in the sample than in the reference standard.

The comparison and/or identification of the presence or absence of a concentration difference (as described above) can be achieved using methods of statistical analysis. The comparison and/or identification of the presence or absence of a difference in intensity of a chemical shift region (as described above) can be achieved using methods of statistical analysis. In one embodiment, a method of statistical analysis suitable for use in the present invention includes orthogonal partial least squares discriminate analysis (OPLS-DA).

Identifying a higher or lower concentration of a metabolite or intensity of a chemical shift region relative to the same metabolite or chemical shift region (respectively) in/of a reference standard preferably means identifying a statistically significant higher or lower concentration or intensity. Identifying the same concentration of a metabolite or intensity of a chemical shift region relative to the same metabolite or chemical shift region (respectively) in/of a reference standard preferably means identifying no statistically significant concentration difference or difference in intensity (respectively). More preferably, identifying the same concentration of a metabolite or intensity of a chemical shift region relative to the same metabolite or chemical shift region (respectively) in/of a reference standard preferably means identifying no concentration difference or difference in intensity (respectively).

It is particularly preferred that when carrying out the methods for confirming that an MS patient is suffering a relapse that at least one reference standard is a non-relapse reference standard. The term “non-relapse reference standard” means a reference standard that is representative of a MS patient (preferably RRMS patient) that is not suffering a relapse (for example, is not suffering one or more symptoms of a relapse). For example, a “non-relapse reference standard” can correspond to the concentration of the metabolite (under comparison) in a sample from a MS patient (preferably RRMS patient) that is not suffering a relapse (for example, is not suffering one or more symptoms of a relapse). In other words, “non-relapse reference standard” is preferably a reference standard that is representative of a MS patient (preferably RRMS patient) that is in remission.

The term “non-relapse reference standard” may be a reference standard that is representative of a MS patient (preferably RRMS patient) that has not suffered a relapse for ≥1, ≥2, ≥4, ≥6, ≥8, ≥10, ≥12, ≥14, ≥16, ≥18, ≥20, ≥22, ≥24 or ≥26 months. The term “non-relapse reference standard” may be a reference standard that is representative of a MS patient (preferably RRMS patient) that has not suffered a relapse for ≥1 month to <6 months. In other words, the term “non-relapse reference standard” may be a reference standard that is representative of a MS patient (preferably RRMS patient) that has been in remission for ≥1 month to <6 months.

The term “non-relapse reference standard” may be a reference standard that is representative of a MS patient (preferably RRMS patient) that has not suffered a relapse for ≥6 months to <24 months. In other words, the term “non-relapse reference standard” may be a reference standard that is representative of a MS patient (preferably RRMS patient) that has been in remission for ≥6 months to <24 months.

Suitably, the term “non-relapse reference standard” may be a reference standard that is representative of a MS patient (preferably RRMS patient) that has not suffered a relapse for ≥24 months. In other words, the term “non-relapse reference standard” may be a reference standard that is representative of a MS patient (preferably RRMS patient) that has been in remission for ≥24 months.

The patient may be a patient that has been confirmed to have multiple sclerosis (e.g. confirmed by alternative methodology known in the art).

Preferably, a “non-relapse reference standard” is representative of a MS patient that does not have any other (non-MS) diseases. Thus, a “non-relapse reference standard” may be a reference standard that has been obtained from a MS patient that does not have (and/or has not had) a non-MS disease (or that did not have a non-MS disease when the reference standard was obtained).

Additionally or alternatively, a “non-relapse reference standard” may be representative of a MS patient that is suffering a symptom that is not associated with the course of the MS disease, but rather is caused by an alternative condition or stimulus such as a fever, infection, stress and/or hot weather. Increased body temperature (e.g. from a fever, over-exercising, hot tub/sauna), infection even in the absence of fever (e.g. the flu, urinary tract, sinus, skin infections) trauma, surgery, new medications, other medical conditions (e.g. high blood sugar in diabetics, for example) and psychological stress may be the cause of a pseudo-relapse.

For example, a “non-relapse reference standard” may be representative of a MS patient that is suffering a “pseudo-relapse” (or a pseudoexacerbation). When the term “pseudo-relapse” is used here (and/or e.g. by physicians), this preferably refers to worsened neurologic symptoms; however, the underlying cause of the worsening is preferably not from new immune system activity or inflammation (e.g. as part of the MS disease), but rather from the damage that has occurred from previous inflammation. Suitably, a pseudo-relapse may not be associated with an active MS lesion on MRI.

In one embodiment, a relapse (or poor prognosis thereof) is confirmed when the concentration of one or more metabolite(s) selected from: lysine, asparagine, glucose, and a lipoprotein having a βCH₂ group is higher in the sample relative to the reference standard, wherein the reference standard is a non-relapse reference standard.

In one embodiment, a relapse (or poor prognosis thereof) is confirmed when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard. In one embodiment, a relapse (or poor prognosis thereof) is confirmed when the concentration of one or more metabolite(s) selected from: phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard.

Preferably, relapse (or poor prognosis thereof) is confirmed when the concentration of one or more metabolite(s) selected from: lysine, asparagine, glucose, and a lipoprotein having a βCH₂ group is higher in the sample relative to the reference standard, wherein the reference standard is a non-relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard.

In one embodiment, a relapse (or poor prognosis thereof) is confirmed when the concentration of lysine is higher in the sample relative to the reference standard, wherein the reference standard is a non-relapse reference standard; and when the concentration of one or more metabolite(s) selected from: asparagine, glucose, and a lipoprotein having a βCH₂ group is higher in the sample relative to the reference standard, wherein the reference standard is a non-relapse reference standard. In one embodiment, a relapse (or poor prognosis thereof) is confirmed when the concentration lysine of is higher in the sample relative to the reference standard, wherein the reference standard is a non-relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard.

In one embodiment, a relapse (or poor prognosis thereof) is confirmed when the concentration of asparagine is higher in the sample relative to the reference standard, wherein the reference standard is a non-relapse reference standard; and when the concentration of one or more metabolite(s) selected from: lysine, glucose, and a lipoprotein having a βCH₂ group is higher in the sample relative to the reference standard, wherein the reference standard is a non-relapse reference standard. In one embodiment, a relapse (or poor prognosis thereof) is confirmed when the concentration of asparagine is higher in the sample relative to the reference standard, wherein the reference standard is a non-relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard.

In one embodiment, a relapse (or poor prognosis thereof) is confirmed when the concentration of leucine is lower in the sample relative to the reference standard, wherein the reference standard is a non-relapse reference standard; and when the concentration of one or more metabolite(s) selected from: phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is lower in the sample relative to the reference standard, wherein the reference standard is a non-relapse reference standard. In one embodiment, a relapse (or poor prognosis thereof) is confirmed when the concentration of leucine is lower in the sample relative to the reference standard, wherein the reference standard is a non-relapse reference standard; and when the concentration of one or more metabolite(s) selected from: lysine, asparagine, glucose, and a lipoprotein having a βCH₂ group is higher in the sample relative to the reference standard, wherein the reference standard is a non-relapse reference standard.

Advantageously, when the reference standard is a non-relapse reference standard, one or more of the following metabolite(s) find particular utility in confirming a relapse: lysine, asparagine and leucine.

In one embodiment, a relapse (or poor prognosis thereof) is not confirmed when the concentration of one or more metabolite(s) selected from: lysine, asparagine, glucose, and a lipoprotein having a βCH₂ group is the same or lower in the sample relative to the reference standard, wherein the reference standard is a non-relapse reference standard.

In one embodiment, a relapse (or poor prognosis thereof) is not confirmed when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard. In one embodiment, a relapse (or poor prognosis thereof) is not confirmed when the concentration of one or more metabolite(s) selected from: phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard.

Preferably, a relapse (or poor prognosis thereof) is not confirmed when the concentration of one or more metabolite(s) selected from: lysine, asparagine, glucose, and a lipoprotein having a βCH₂ group is the same or lower in the sample relative to the reference standard, wherein the reference standard is a non-relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard.

In one embodiment, a relapse (or poor prognosis thereof) is not confirmed when the concentration of lysine is the same or lower in the sample relative to the reference standard, wherein the reference standard is a non-relapse reference standard; and the concentration of one or more metabolite(s) selected from: asparagine, glucose, and a lipoprotein having a βCH₂ group is the same or lower in the sample relative to the reference standard, wherein the reference standard is a non-relapse reference standard. In one embodiment, a relapse (or poor prognosis thereof) is not confirmed when the concentration of lysine is the same or lower in the sample relative to the reference standard, wherein the reference standard is a non-relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard.

In one embodiment, a relapse (or poor prognosis thereof) is not confirmed when the concentration of asparagine is the same or lower in the sample relative to the reference standard, wherein the reference standard is a non-relapse reference standard; and the concentration of one or more metabolite(s) selected from: lysine, glucose, and a lipoprotein having a βCH₂ group is the same or lower in the sample relative to the reference standard, wherein the reference standard is a non-relapse reference standard. In one embodiment, a relapse (or poor prognosis thereof) is not confirmed when the concentration of asparagine is the same or lower in the sample relative to the reference standard, wherein the reference standard is a non-relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard.

In one embodiment, a relapse (or poor prognosis thereof) is not confirmed when the concentration of leucine is the same or higher in the sample relative to the reference standard, wherein the reference standard is a non-relapse reference standard; and when the concentration of one or more metabolite(s) selected from: phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or higher in the sample relative to the reference standard, wherein the reference standard is a non-relapse reference standard. In one embodiment, a relapse (or poor prognosis thereof) is not confirmed when the concentration of leucine is the same or higher in the sample relative to the reference standard, wherein the reference standard is a non-relapse reference standard; and when the concentration of one or more metabolite(s) selected from: lysine, asparagine, glucose, and a lipoprotein having a βCH₂ group is the same or lower in the sample relative to the reference standard, wherein the reference standard is a non-relapse reference standard.

In one embodiment, a relapse (or poor prognosis thereof) is confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard.

In one embodiment, a relapse (or poor prognosis thereof) is confirmed when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is lower in the sample relative to the reference standard, wherein the reference standard is a non-relapse reference standard.

Preferably, a relapse (or poor prognosis thereof) is confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard; and when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is lower in the sample relative to the reference standard, wherein the reference standard is a non-relapse reference standard.

Advantageously, one or more of the following chemical shift region(s) find particular utility in a method of the invention: 1.37-1.55 ppm, 1.65-1.75 ppm, 1.83-1.94 ppm, 3.00-3.05 ppm, 2.80-3.00 ppm, 3.96-4.02 ppm, 0.94-0.98 ppm, 1.62-1.78 ppm, 3.70-3.79 ppm, 0.92-0.97 ppm, 1.00-1.03 ppm, 1.22-1.28 ppm, 1.43-1.51 ppm, 1.94-2.01 ppm, and 3.65-3.68 ppm (preferably 1.37-1.55 ppm, 1.65-1.75 ppm, 1.83-1.94 ppm, 3.00-3.05 ppm, 2.80-3.00 ppm, 3.96-4.02 ppm, 0.94-0.98 ppm, 1.62-1.78 ppm, and/or 3.70-3.79 ppm).

In one embodiment, a relapse (or poor prognosis thereof) is not confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard.

In one embodiment, a relapse (or poor prognosis thereof) is not confirmed when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard.

In one embodiment, a relapse (or poor prognosis thereof) is not confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard; and when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard.

In one embodiment, at least one relapse reference standard may be used in a method of the invention. The term “relapse reference standard” means a reference standard that is representative of a MS patient (preferably RRMS patient) that is suffering a relapse. For example, a “relapse reference standard” may correspond to the concentration of the metabolite (under comparison) in sample from a MS patient (preferably RRMS patient) that is suffering a relapse. In other words, “relapse reference standard” is preferably a reference standard that is representative of a MS patient (preferably RRMS patient) that is not in remission.

A “relapse reference standard” may be a reference standard that has been obtained from a MS subject that is suffering a relapse (or was suffering a relapse at the time the reference standard was obtained). In other words, a “relapse reference standard” may be a reference standard that has been obtained from a MS subject that is not in remission (or was not in remission at the time the reference standard was obtained).

The term “relapse reference standard” may be a reference standard that is representative of a MS patient (preferably RRMS patient) that has been suffering a relapse for ≤1 month. In other words, the term “relapse reference standard” may be a reference standard that is representative of a MS patient (preferably RRMS patient) that has not been in remission for ≤1 month. The term “relapse reference standard” may be a reference standard that is representative of a MS patient (preferably RRMS patient) that has been suffering a relapse for <1 month. In other words, the term “relapse reference standard” may be a reference standard that is representative of a MS patient (preferably RRMS patient) that has not been in remission for <1 month.

A “relapse reference standard” may be a reference standard that has been obtained from a MS subject that has been suffering a relapse for ≤1 month (or was suffering a relapse for ≤1 month at the time the reference standard was obtained). In other words, a “relapse reference standard” may be a reference standard that has been obtained from a MS subject that has not been in remission for ≤1 month (or was not in remission for ≤1 month at the time the reference standard was obtained). A “relapse reference standard” may be a reference standard that has been obtained from a MS subject that has been suffering a relapse for <1 month (or was suffering a relapse for <1 month at the time the reference standard was obtained). In other words, a “relapse reference standard” may be a reference standard that has been obtained from a MS subject that has not been in remission for 51 month (or was not in remission for ≤1 month at the time the reference standard was obtained).

In one embodiment, a relapse (or poor prognosis thereof) is confirmed when the concentration of one or more metabolite(s) selected from: lysine, asparagine, glucose, and a lipoprotein having a βCH₂ group is the same or higher in the sample relative to the reference standard, wherein the reference standard is a relapse reference standard.

In one embodiment, a relapse (or poor prognosis thereof) is confirmed when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard.

Preferably, a relapse (or poor prognosis thereof) is confirmed when the concentration of one or more metabolite(s) selected from: lysine, asparagine, glucose, and a lipoprotein having a βCH₂ group is the same or higher in the sample relative to the reference standard, wherein the reference standard is a relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard.

In one embodiment, a relapse (or poor prognosis thereof) is confirmed when the concentration of lysine is the same or higher in the sample relative to the reference standard, wherein the reference standard is a relapse reference standard; and the concentration of one or more metabolite(s) selected from: asparagine, glucose, and a lipoprotein having a βCH₂ group is the same or higher in the sample relative to the reference standard, wherein the reference standard is a relapse reference standard. In one embodiment, a relapse (or poor prognosis thereof) is confirmed when the concentration of lysine is the same or higher in the sample relative to the reference standard, wherein the reference standard is a relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard.

In one embodiment, a relapse (or poor prognosis thereof) is confirmed when the concentration of asparagine is the same or higher in the sample relative to the reference standard, wherein the reference standard is a relapse reference standard; and the concentration of one or more metabolite(s) selected from: lysine, glucose, and a lipoprotein having a βCH₂ group is the same or higher in the sample relative to the reference standard, wherein the reference standard is a relapse reference standard. In one embodiment, a relapse (or poor prognosis thereof) is confirmed when the concentration of asparagine is the same or higher in the sample relative to the reference standard, wherein the reference standard is a relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is the same or lower in the sample relative to the reference standard, wherein the reference standard is a relapse reference standard.

In one embodiment, methods of the invention comprise comparing a concentration of two or more metabolite(s) (preferably three or more metabolite(s); even more preferably four or more metabolite(s)) present in a sample obtained from the patient with the concentration of the same two or more metabolite(s) (preferably three or more metabolite(s); even more preferably four or more metabolite(s)) in a reference standard, wherein the two or more metabolite(s) (preferably three or more metabolite(s); even more preferably four or more metabolite(s)) are selected from: lysine, leucine, asparagine and isoleucine; and confirming that the patient is suffering from a relapse (or determining that the patient's prognosis is poor) when:
the concentration of leucine is lower in the sample relative to the reference standard, wherein the reference standard is a non-relapse reference standard; and/or
the concentration of lysine is higher in the sample relative to the reference standard, wherein the reference standard is a non-relapse reference standard; and/or
the concentration of asparagine is higher in the sample relative to the reference standard, wherein the reference standard is a non-relapse reference standard; and/or
the concentration of isoleucine is lower in the sample relative to the reference standard, wherein the reference standard is a non-relapse reference standard; and/or
the concentration of leucine is the same or lower in the sample relative to the reference standard, wherein the reference standard is a relapse reference standard; and/or
the concentration of lysine is the same or higher in the sample relative to the reference standard, wherein the reference standard is a relapse reference standard; and/or
the concentration of asparagine is the same or higher in the sample relative to the reference standard, wherein the reference standard is a relapse reference standard; and/or
the concentration of isoleucine is the same or lower in the sample relative to the reference standard, wherein the reference standard is a relapse reference standard.

In one embodiment, methods of the invention comprise comparing a concentration of four or more metabolite(s) present in a sample obtained from the patient with the concentration of the same four or more metabolite(s) in a reference standard, wherein the four or more metabolite(s) are selected from: lysine, leucine, asparagine and isoleucine; and confirming that the patient is suffering from a relapse (or determining that the patient's prognosis is poor) when:
the concentration of leucine is lower in the sample relative to the reference standard, wherein the reference standard is a non-relapse reference standard; and
the concentration of lysine is higher in the sample relative to the reference standard, wherein the reference standard is a non-relapse reference standard; and
the concentration of asparagine is higher in the sample relative to the reference standard, wherein the reference standard is a non-relapse reference standard; and
the concentration of isoleucine is lower in the sample relative to the reference standard, wherein the reference standard is a non-relapse reference standard; and
the concentration of leucine is the same or lower in the sample relative to the reference standard, wherein the reference standard is a relapse reference standard; and
the concentration of lysine is the same or higher in the sample relative to the reference standard, wherein the reference standard is a relapse reference standard; and
the concentration of asparagine is the same or higher in the sample relative to the reference standard, wherein the reference standard is a relapse reference standard; and
the concentration of isoleucine is the same or lower in the sample relative to the reference standard, wherein the reference standard is a relapse reference standard.

In one embodiment, methods of the invention comprise comparing a concentration of two or more metabolite(s) (preferably three or more metabolite(s); even more preferably four or more metabolite(s)) present in a sample obtained from the patient with the concentration of the same two or more metabolite(s) (preferably three or more metabolite(s); even more preferably four or more metabolite(s)) in a reference standard, wherein the two or more metabolite(s) (preferably three or more metabolite(s); even more preferably four or more metabolite(s)) are selected from: lysine, leucine, asparagine and isoleucine; and not confirming that the patient is suffering from a relapse, or confirming that the patient is not suffering from a relapse (or not determining that the subject's prognosis is poor, or determining that the patient's prognosis is good) when:
the concentration of leucine is the same or higher in the sample relative to the reference standard, wherein the reference standard is a non-relapse reference standard; and/or
the concentration of lysine is the same or lower in the sample relative to the reference standard, wherein the reference standard is a non-relapse reference standard; and/or
the concentration of asparagine is the same or lower in the sample relative to the reference standard, wherein the reference standard is a non-relapse reference standard; and/or
the concentration of isoleucine is the same or higher in the sample relative to the reference standard, wherein the reference standard is a non-relapse reference standard; and/or
the concentration of leucine is higher in the sample relative to the reference standard, wherein the reference standard is a relapse reference standard; and/or
the concentration of lysine is lower in the sample relative to the reference standard, wherein the reference standard is a relapse reference standard; and/or
the concentration of asparagine is lower in the sample relative to the reference standard, wherein the reference standard is a relapse reference standard; and/or
the concentration of isoleucine is higher in the sample relative to the reference standard, wherein the reference standard is a relapse reference standard.

In one embodiment, methods of the invention comprise comparing a concentration of four or more metabolite(s) present in a sample obtained from the patient with the concentration of the same four or more metabolite(s)) in a reference standard, wherein the four or more metabolite(s) are selected from: lysine, leucine, asparagine and isoleucine; and not confirming that the patient is suffering from a relapse, or confirming that the patient is not suffering from a relapse (or not determining that the subject's prognosis is poor, or determining that the patient's prognosis is good) when:
the concentration of leucine is the same or higher in the sample relative to the reference standard, wherein the reference standard is a non-relapse reference standard; and
the concentration of lysine is the same or lower in the sample relative to the reference standard, wherein the reference standard is a non-relapse reference standard; and
the concentration of asparagine is the same or lower in the sample relative to the reference standard, wherein the reference standard is a non-relapse reference standard; and
the concentration of isoleucine is the same or higher in the sample relative to the reference standard, wherein the reference standard is a non-relapse reference standard; and
the concentration of leucine is higher in the sample relative to the reference standard, wherein the reference standard is a relapse reference standard; and
the concentration of lysine is lower in the sample relative to the reference standard, wherein the reference standard is a relapse reference standard; and
the concentration of asparagine is lower in the sample relative to the reference standard, wherein the reference standard is a relapse reference standard; and
the concentration of isoleucine is higher in the sample relative to the reference standard, wherein the reference standard is a relapse reference standard.

In one embodiment, a relapse (or poor prognosis thereof) is not confirmed when the concentration of one or more metabolite(s) selected from: lysine, asparagine, glucose, and a lipoprotein having a βCH₂ group is lower in the sample relative to the reference standard, wherein the reference standard is a relapse reference standard.

In one embodiment, a relapse (or poor prognosis thereof) is not confirmed when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

Preferably, a relapse (or poor prognosis thereof) is not confirmed when the concentration of one or more metabolite(s) selected from: lysine, asparagine, glucose, and a lipoprotein having a βCH₂ group is lower in the sample relative to the reference standard, wherein the reference standard is a relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

In one embodiment, a relapse (or poor prognosis thereof) is not confirmed when the concentration of lysine is lower in the sample relative to the reference standard, wherein the reference standard is a relapse reference standard; and when concentration of one or more metabolite(s) selected from: asparagine, glucose, and a lipoprotein having a βCH₂ group is lower in the sample relative to the reference standard, wherein the reference standard is a relapse reference standard. In one embodiment, a relapse (or poor prognosis thereof) is not confirmed when the concentration of lysine is lower in the sample relative to the reference standard, wherein the reference standard is a relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

In one embodiment, a relapse (or poor prognosis thereof) is not confirmed when the concentration of asparagine is lower in the sample relative to the reference standard, wherein the reference standard is a relapse reference standard; and when the concentration of one or more metabolite(s) selected from: lysine, glucose, and a lipoprotein having a βCH₂ group is lower in the sample relative to the reference standard, wherein the reference standard is a relapse reference standard. In one embodiment, a relapse (or poor prognosis thereof) is not confirmed when the concentration of asparagine is lower in the sample relative to the reference standard, wherein the reference standard is a relapse reference standard; and when the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard.

In one embodiment, a relapse (or poor prognosis thereof) is not confirmed when the concentration of leucine is higher in the sample relative to the reference standard, wherein the reference standard is a relapse reference standard; and the concentration of one or more metabolite(s) selected from: phenylalanine, β-hydroxybutyrate, myo-inositol, a lipoprotein having a —CH₃ group of an HDL and/or LDL, a lipoprotein having a —CH₃ group of a VLDL, a lipoprotein having —(CH₂)ₙ group of an HDL and/or LDL, and an N-acetylated glycoprotein is higher in the sample relative to the reference standard, wherein the reference standard is a relapse reference standard. In one embodiment, a relapse (or poor prognosis thereof) is not confirmed when the concentration of leucine is higher in the sample relative to the reference standard, wherein the reference standard is a relapse reference standard; and when the concentration of one or more metabolite(s) selected from: lysine, asparagine, glucose, and a lipoprotein having a βCH₂ group is lower in the sample relative to the reference standard, wherein the reference standard is a relapse reference standard.

In one embodiment, a relapse (or poor prognosis thereof) is confirmed when the concentration of leucine is the same or lower in the sample relative to the reference standard, wherein the reference standard is a relapse reference standard; and the concentration of one or more metabolite(s) selected from: lysine, asparagine, glucose, and a lipoprotein having a βCH₂ group is the same or higher in the sample relative to the reference standard, wherein the reference standard is a relapse reference standard.

In one embodiment, a relapse (or poor prognosis thereof) is confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard.

In one embodiment, relapse (or poor prognosis thereof) when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard.

Preferably, a relapse (or poor prognosis thereof) is confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard; and when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard.

In one embodiment, a relapse (or poor prognosis thereof) is not confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard.

In one embodiment, a relapse (or poor prognosis thereof) is not confirmed when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a relapse reference standard.

Preferably, a relapse (or poor prognosis thereof) is not confirmed when the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard; and when the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a relapse reference standard.

The methods of the invention may further comprise comparing a concentration of isoleucine present in a sample obtained from a subject with the concentration of isoleucine in a reference standard. A relapse (or poor prognosis thereof) may be confirmed when the concentration of isoleucine is lower in the sample relative to the reference standard, wherein the reference standard is a non-relapse reference standard. A relapse (or poor prognosis thereof) may also be confirmed when the concentration of isoleucine is the same or lower in the sample relative to the reference standard, wherein the reference standard is a relapse reference standard. In one embodiment, a relapse (or poor prognosis thereof) is not confirmed when the concentration of isoleucine is the same or higher in the sample relative to the reference standard, wherein the reference standard is a non-relapse reference standard. In one embodiment, a relapse (or poor prognosis thereof) is not confirmed when the concentration of isoleucine is higher in the sample relative to the reference standard, wherein the reference standard is a relapse reference standard.

The methods of the invention may further comprise comparing an intensity of a further one or more chemical shift region of a ¹H-NMR spectrum of a sample obtained from a subject with the intensity of the same one or more chemical shift region of a ¹H-NMR reference standard, wherein the chemical shift region is 0.92-0.97 ppm, 1.00-1.03 ppm, 1.22-1.28 ppm, 1.43-1.51 ppm, 1.94-2.01 ppm, and/or 3.65-3.68 ppm (preferably 0.92-0.97 ppm, 1.00-1.03 ppm, 1.22-1.28 ppm, 1.43-1.51 ppm, 1.94-2.01 ppm, and 3.65-3.68 ppm). A relapse (or poor prognosis thereof) may be confirmed when the intensity of said chemical shift region of the ¹H-NMR spectrum is lower in the sample relative to the reference standard, wherein the reference standard is a non-relapse reference standard. A relapse (or poor prognosis thereof) may also be confirmed when the intensity of said chemical shift region of the ¹H-NMR spectrum is the same or lower in the sample relative to the reference standard, wherein the reference standard is a relapse reference standard. In one embodiment, a relapse (or poor prognosis thereof) is not confirmed when the intensity of said chemical shift region of the ¹H-NMR spectrum is the same or higher in the sample relative to the reference standard, wherein the reference standard is a non-relapse reference standard. In one embodiment, a relapse (or poor prognosis thereof) is not confirmed when the intensity of said chemical shift region of the ¹H-NMR spectrum is higher in the sample relative to the reference standard, wherein the reference standard is a relapse reference standard.

The methods of the invention may further comprise comparing a concentration of NfL present in a sample obtained from a subject with the concentration of NfL in a reference standard. A relapse (or poor prognosis thereof) may be confirmed when the concentration of NfL is higher in the sample relative to the reference standard, wherein the reference standard is a non-relapse reference standard. A relapse (or poor prognosis thereof) may also be confirmed when the concentration of NfL is the same or higher in the sample relative to the reference standard, wherein the reference standard is a relapse reference standard. In one embodiment, a relapse (or poor prognosis thereof) is not confirmed when the concentration of NfL is the same or lower in the sample relative to the reference standard, wherein the reference standard is a non-relapse reference standard. In one embodiment, a relapse (or poor prognosis thereof) is not confirmed when the concentration of NfL is lower in the sample relative to the reference standard, wherein the reference standard is a relapse reference standard.

In one embodiment, the method of the invention further comprises recording the output of at least one step on a data-storage medium. By way of example, the methods of the present invention can generate data relating to the subject, such data being recordable on a data-storage medium (for example, a form of computer memory such as a hard disk, compact disc, floppy disk, or solid state drive). Such data can comprise (or consist of) data relating to the concentration in a sample (from said subject) of any of one or more metabolites (as described herein) and/or data relating to the intensity in a sample (from said subject) of any of one or more chemical shift regions (as described) herein.

In one aspect the invention provides a data-storage medium, comprising data obtained by a method according to the present invention.

In one aspect, the invention provides a computer program product comprising program instructions to cause a processor to perform a method according to the invention.

In another aspect, the invention provides a device for use in a method of the invention, wherein said device is capable of performing the step of identifying: a concentration (e.g. a concentration difference) of one or more metabolites in the sample when compared to the reference standard and/or an intensity (e.g. a difference in intensity) of one or more chemical shift regions of a ¹H-NMR spectrum of a sample obtained from a subject when compared to the reference standard.

In one aspect, the invention provides a method of treating a MS patient suffering from a relapse, the method comprising:
(a) obtaining the results of a method according to the invention; and
(b) administering a therapy suitable for treating relapse (or a symptom of relapse) when a relapse is confirmed (e.g. diagnosed); and
(c) optionally administering a different therapy when a relapse is not confirmed (e.g. diagnosed).

Treatment of relapse (or symptom thereof) may be carried out using any suitable therapeutic for a relapse (or symptom thereof) known in the art. A suitable therapy is preferably a disease modifying therapy/therapies for MS, which reduces the number of relapses, and/or which may delay progression of disability. For example, therapy may include an injectable medication, such as Avonex (interferon beta-1a); Betaseron (interferon beta-1b); Copaxone (glatiramer acetate), Extavia (interferon beta-1b), Glatiramer Acetate Injection (glatiramer acetate, generic equivalent of Copaxone 20 mg and 40 mg doses), Glatopa (glatiramer acetate, generic equivalent of Copaxone 20 mg and 40 mg doses), Kesimpta® (ofatumumab), Plegridy (peginterferon beta-1a), and/or Rebif (interferon beta-1a); an oral medication, such as Aubagio (teriflunomide), Bafiertam (monomethyl fumarate), Gilenya (fingolimod), Mavenclad (cladribine), Mayzent (siponimod), Tecfidera (dimethyl fumarate), Vumerity (diroximel fumarate), oral methylprednisolone, and/or Zeposia (ozanimod); and/or an infused medication, such as Lemtrada (alemtuzumab), Novantrone (mitoxantrone), Ocrevus (ocrelizumab), intravenous methylprednisolone, and/or Tysabri (natalizumab). The treatment may additionally or alternatively comprise include autologous hematopoietic stem cell transplantation (AHSCT).

The term “disorder” as used herein also encompasses a “disease”. In one embodiment, the disorder is a disease. The disorder treated in accordance with the invention is suitably MS, wherein the MS patient is suffering a relapse.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment.

The term “treat” or “treating” as used herein refers to the disorder and/or a symptom thereof.

Therefore, a therapeutic may be administered to a subject in a therapeutically effective amount or a prophylactically effective amount.

A “therapeutically effective amount” is any amount of a therapeutic formulation, which when administered alone or in combination to a subject for treating said disorder (or a symptom thereof) is sufficient to effect such treatment of the disorder, or symptom thereof.

A “prophylactically effective amount” may be any amount of a therapeutic formulation that, when administered alone or in combination to a subject inhibits or delays the onset or reoccurrence of a disorder (or a symptom thereof). In some embodiments, the prophylactically effective amount prevents the onset or reoccurrence of a disorder entirely. “Inhibiting” the onset means either lessening the likelihood of a disorder's onset (or symptom thereof), or preventing the onset entirely. Preferably, a “prophylactically effective amount” is any amount of a therapeutic formulation that, when administered alone or in combination to a subject inhibits or delays the onset or reoccurrence of a relapse (or a symptom thereof). In some embodiments, the prophylactically effective amount prevents the onset or reoccurrence of a relapse entirely. “Inhibiting” the onset means either lessening the likelihood of a relapse's onset (or symptom thereof), or preventing the onset entirely.

Administration may be by any route known in the art and will typically be dependent on the nature of the therapeutic to be administered. For example, a therapeutic may be administered orally or parenterally. Methods of parenteral delivery include topical, intra-arterial, intramuscular, subcutaneous, intramedullary, intrathecal, intra-ventricular, intravenous, intraperitoneal, or intranasal administration.

Embodiments related to the various methods of the invention are intended to be applied equally to other methods, therapeutic uses or methods, the data storage medium or device, the computer program product, and vice versa.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. The headings provided herein are not limitations of the various aspects or embodiments of this disclosure.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a metabolite” includes a plurality of such candidate agents and reference to “the metabolites” includes reference to one or more metabolites and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples, in which:

FIG. 1 shows representative OPLS-DA models generated from CPMG data of ln R vs. LR≥24 M patients. (A) Representative OPLS-DA scores plot (ln R=red circles, LR≥24 M=green triangles). (B) Box plots of predictive accuracies from the OPLS-DA models of ln R vs. LR≥24 M patients, against random class assignment. ****indicates p<0.0001 by Kolmogorov-Smirnov test.

FIG. 2 shows a bar graphs demonstrating the fold change in predictive accuracies of the OPLS-DA models of the different patient groups with respect to the reference comparator, i.e. LR≥24 M patients. The fold change of random chance is 1.0 as indicated by the dashed horizontal line. ****indicates p<0.0001 by post-hoc Bonferroni correction.

FIG. 3 shows a VIP score ranking plot obtained from the OPLS-DA models of ln R vs. LR≥24 M patients. The dashed red line indicates the VIP score threshold of 1.35, before a ‘drop-off’ in VIP score.

FIG. 4 shows representative OPLS-DA models generated from AXINON® lipoFIT® data of ln R vs. LR≥24 M patients. (A) Representative OPLS-DA scores plot (ln R=red circles, LR≥24 M=green triangles). (B) Box plots of predictive accuracies from the OPLS-DA models of ln R vs. LR≥24 M patients, against random class assignment. ****indicates p<0.0001 by Kolmogorov-Smirnov test.

FIG. 5 shows VIP score ranking plot obtained from the OPLS-DA models of IR vs. LR≥2 years patients identifies isoleucine and leucine as the top two discriminatory metabolites.

FIG. 6 shows BCAA resonances in a CPMG spectrum. The zoom in panel shows that the spectral resonances overlap (e.g. isoleucine overlap with the broad methyl lipoprotein resonance; leucine resonance overlap with this broad lipoprotein signal, to a lesser extent); any masking due to overlap was overcome by targeted metabolomics (e.g. AXINON® lipoFIT® spectra data acquisition).

FIG. 7 shows a workflow schematic illustrating the selection of metabolites for ANOVA. *indicates metabolites detected by targeted metabolomics.

FIG. 8 shows bar graphs demonstrating significant metabolites on one-way ANOVA. (A and B) Lysine and asparagine levels were higher within ln R patients compared to LR≥24 M patients and decreased with time away from relapse. (C and D) In contrast, isoleucine and leucine concentrations were observed to be lower during relapses and increased with time away from relapse. **indicates p<0.01 and *indicates p<0.05 by post-hoc Holm-Sidak's test, with LR≥24 M patients as the reference comparator.

FIG. 9 shows ROC analysis of the four metabolite biomarkers (A-D) individually and (E) in combination.

FIG. 10 shows results from investigating the discriminatory ability of serum NfL levels across the patient groups. (A) One-way ANOVA showed that ln R patients had higher levels of serum NfL compared to LR≥24 M patients. ****indicates p<0.0001 by post-hoc Holm-Sidak's test, with LR≥24 M patients as the reference comparator. (B) ROC analysis of serum NfL. (C) ROC analysis using a combination of lysine, asparagine, isoleucine, leucine and NfL.

FIG. 11 shows results from investigating paired relapse-remission levels for the (A-D) four metabolite biomarkers, and for (E) serum NfL. *indicates p<0.05 on paired t-test.

FIG. 12 shows results from multivariate approaches to combine the four metabolites as a composite biomarker. (A) ROC analysis after multivariable logistic regression using the four metabolites. (B) Multivariable ROC analysis with the addition of serum NfL to the four metabolites. (C) OPLS-DA scores plot of ln R vs. LR≥24 M patients (ln R=circles, LR≥24 M=triangles), minus the in relapse data points of the nine patients with paired relapse-remission samples. (D) Insertion of data from paired relapse-remission samples into the OPLS-DA scores plot as a predictive set (relapse=diamonds within circles, remission=diamonds not within circles). Arrows indicate paired samples.

FIG. 13 shows results from investigating paired remission-relapse metabolites and serum NfL levels as predictive biomarkers of future relapses (A) Isoleucine, (B) leucine, and (C) serum NfL.

FIG. 14 shows results of analysis of plasma lipoprotein subclass particle number (-p), size, and cholesterol content (-c) of non-responders (defined as clinical relapse during 6 month follow-up) and responders (defined as no clinical relapse during follow-up) to first-line MS therapy using the AXINON® lipoFIT® platform. A significant increase in LDL particle number and cholesterol content was observed in the responder group relative to the non-responders.

FIG. 15 shows results of OPLS-DA NMR metabolomics analysis of plasma samples from a cohort of 44 people with MS receiving glatiramer acetate treatment provides additional evidence that plasma metabolites can distinguish between treatment responders (defined as no clinical relapse during 6 month follow up) and non-responders (defined as clinical relapse during 6 month follow up). A) A representative OPLS-DA scores plot (from the ensemble of 1000 cross-validated models generated) illustrates excellent discrimination between classes. B) A subset of the model validation metrics are provided including the predictive accuracy, sensitivity, specificity and cumulative Q₂ to illustrate statistical significance of the model, robustness, and validation on independent data. Each box plot presents 1000 predictive values determined on randomly selected test sets. A permutation test generates the null distribution (1000 models) in order to validate the model and confirm discrimination has not occurred by chance. Kolmogorov-Smirnov test p-values<0.001 are represented by ***.

FIG. 16 shows results of unsupervised analysis of baseline blood samples from people with MS who go on to respond/not respond to 1st line therapy spontaneously clusters. This pilot data illustrates that significant differences in the blood metabolite profile exist which are predictive of response to 1st line treatments.

### EXAMPLES

Materials and Methods

Patients

RRMS patients (under the MET cohort collection) were prospectively recruited from the John Radcliffe Hospital, Oxford University Hospital Trust. All patients fulfilled the 2017 revisions to the McDonald criteria for MS. Patients suspected to be having a relapse was first triaged by an experienced MS nurse via phone consultation and those suspected to have a relapse were then seen at the ‘acute relapse’ clinic. Relapse status was established by MS neurologists and defined clinically (e.g. without the need for additional MRI confirmation) in accordance to the McDonald criteria, i.e. a monophasic clinical episode with patient-reported symptoms and objective findings typical of MS, reflecting a focal or multifocal inflammatory demyelinating event in the CNS, developing acutely or subacutely, with a duration of at least 24 hours, with or without recovery, and in the absence of fever or infection. Patients with “pseudo-relapses” were excluded. This was performed by systemic review of infective symptoms, temperature measurement and urine dipstick assessment to rule out urinary tract infections. Detailed information on smoking, alcohol intake, time from last meal and type of food for last meal were also collected at the time of recruitment.

Patients were divided into four groups according to the interval between their last relapse to blood sampling: (1) in relapse, defined as <1 month from the onset of relapse; (2) last relapse (LR)≥1 month to <6 months ago; (3) LR≥6 months to <24 months ago; and (4) LR≥24 months ago. These groups are henceforth referred to as ‘ln R’, ‘LR 1-6 M’, ‘LR 6-24 M’ and ‘LR≥24 M’ respectively.

In more detail, a total of two hundred and one RRMS patients were included in this study: In R (n=38), LR 1-6 M (n=28), LR 6-24 M (n=34), and LR≥24 M (n=101). Demographic and clinical characteristics are shown in Table 1 below:

TABLE 1
Demographic and clinical details of study population.

| | | | | | | | | | p value |
|---|---|---|---|---|---|---|---|---|---|
| | In R | | LR 1-6 M | | LR 6-24 M | | LR ≥24 M | | across |
| | (n = 38) | | (n = 28) | | (n = 34) | | (n = 101) | | groups |
| Age in years, mean (SD) | 38.3 | (9.5)* | 38.7 | (7.0)† | 43.5 | (9.7) | 44.2 | (9.9)*† | 0.002 |
| Female, No. (%) | 27 | (71.1) | 23 | (82.1) | 22 | (64.7) | 73 | (72.3) | 0.503 |
| White ethnicity, No. (%) | 37 | (97.4) | 25 | (89.3) | 31 | (91.2) | 92 | (91.1) | 0.589 |
| Recent/current steroid use, No. (%) | 5 | (13.2)* | 0 | (0.0) | 1 | (2.9) | 1 | (1.0)* | 0.011 |
| DMT use, No. (%) | 18 | (47.4)* | 12 | (42.9)† | 20 | (58.8) | 75 | (74.3)*† | 0.002 |
| Alemtuzumab | 1 | (5.6) | 1 | (8.3) | 1 | (5.0) | 2 | (2.7) | |
| Dimethyl fumarate | 6 | (33.3) | 2 | (16.7) | 6 | (30.0) | 13 | (17.3) | |
| Fingolimod | 2 | (11.1) | 2 | (16.7) | 2 | (10.0) | 10 | (13.3) | |
| Glatiramer acetate | 6 | (33.3) | 5 | (41.7) | 7 | (35.0) | 25 | (33.3) | |
| Interferons | 1 | (5.6) | 0 | (0.0) | 3 | (15.0) | 18 | (24.0) | |
| Natalizumab | 1 | (5.6) | 2 | (16.7) | 1 | (5.0) | 5 | (6.7) | |
| Teriflunomide | 1 | (5.6) | 0 | (0.0) | 0 | (0.0) | 2 | (2.7) | |
| EDSS, median (range) | 3.3 | (1-7)*^{ϕ}^{θ} | 2.5 | (1-6.5)^{ϕ} | 2.3 | (0-8.5)^{θ} | 2.0 | (0-7)* | <0.001 |
| Disease duration in years, median | 11.1 | (0.73-28.7) | 7.5 | (0.19-28.3)† | 4.4 | (0.54-28.5)^{φ} | 12.1 | (2.3-47.3)†^{φ} | <0.001 |
| (range) | | | | | | | | | |
| No comorbidities, No. (%) | 16 | (42.1) | 10 | (35.7) | 8 | (23.5) | 42 | (41.6) | 0.271 |
| Presence of new T₂ lesion/s within | 5/12 | (41.7) | 7/8 | (87.5)† | 4/9 | (44.4) | 3/22 | (13.6)† | 0.002 |
| last 1 year, referenced to a baseline | | | | | | | | | |
| scan done ≤1 year apart, No. (%) | | | | | | | | | |
| Presence of GAD-enhancing | 5/16 | (31.3) | 7/11 | (63.6)† | 4/12 | (33.3) | 1/21 | (4.8)† | 0.003 |
| lesion/s within last 1 year, No. (%) | | | | | | | | | |
| BMI, median (range) | 26.5 | (20-49) | 25.0 | (19-38.7) | 27.0 | (19.8-57.4) | 24.8 | (15-42) | 0.081 |
| Current smoker, No. (%) | 5 | (13.2) | 5 | (17.9) | 3 | (8.8) | 12 | (11.9) | 0.751 |
| Alcohol intake in units/week, | 0 | (0-16) | 1.5 | (0-35) | 0 | (0-18) | 1 | (0-24) | 0.394 |
| median (range) | | | | | | | | | |
| Time from last meal in hours, | 3.7 | (1.3-18.5) | 3.6 | (0.8-20.9) | 3.6 | (0.4-16.3) | 3.3 | (0.9-16.7) | 0.319 |
| median (range) | | | | | | | | | |

p values within the right most column indicates differences across the four groups of patients. Symbols indicate p < 0.05 for pair-wise comparison after post-hoc correction.
*In R vs. LR ≥24 M; †LR 1-6 M vs. LR ≥24 M; ^{ϕ}In R vs. LR 1-6 M; ^{θ}In R vs. LR 6-24 M; ^{φ}LR 6-24 M vs. LR ≥24 M.

Blood Collection, Serum Processing

The detailed protocol, as described in the paragraph below, was used for pre-analytical sample handling. Two hundred and fifty microlitres of serum was used for global metabolomics while 750 uL of serum from the same blood sample was used for targeted metabolomics.

During venepuncture, blood was collected into red-top (BD™ Vacutainer™ 367837) and green-top lithium-heparin tubes (BD™ Vacutainer™ 367375) for serum and plasma collection respectively. The blood sample-handling protocol used is consistent with those frequently employed in the metabolomics literature and involved the following steps (Bervoets et al., 2015; Yin et al., 2015). Once collected, blood was left to stand for 30 minutes (to give sufficient time for clot formation in the case of serum) at room temperature after which it was centrifuged at 1,300 g for 10 minutes at room temperature using a benchtop centrifuge (Medifuge™, Thermo Fisher Scientific Inc.) for erythrocyte separation to obtain serum/plasma. The serum/plasma was then immediately aliquoted into polypropylene cryotubes (Crystal Clear™, STARLAB UK Ltd) and stored at −80° C. until NMR sample preparation.

NMR Sample Preparation

For NMR sample preparation, human serum/plasma was thawed at room. Two hundred microlitres was then diluted with 400 μL of 75 mM sodium phosphate buffer, to make up a total volume of 600 μL. The sodium phosphate buffer was prepared by dissolving 62.5 mM of anhydrous sodium phosphate dibasic powder (CAS number 7558794) and 12.5 mM of anhydrous sodium phosphate monobasic powder (CAS number 7558807) in deuterium oxide (D₂O) (CAS number 7789200), giving a final pH of 7.4 (all three reagents were obtained from Sigma-Aldrich, Dorset, UK). D₂O was used as the NMR solvent for all NMR experiments.

In more detail, the NMR experiments were performed using a 700-MHz Bruker (Bruker BioSpin Gmbh, Rheinstetten, Germany) AVIII spectrometer operating at 16.4 T equipped with a ¹H [¹³C/¹⁵N] TCI cryoprobe at the Department of Chemistry, University of Oxford. Sample temperature was regulated at 310 K.

For all serum/plasma NMR samples used, ¹H NMR spectra were first acquired using a 1D NOESY presaturation scheme for suppression of the water resonance with a 2 second (s) presaturation, 8 data collections, an acquisition time of 1.5 s, and a fixed receiver gain. Following this, a spin-echo Carr-Purcell-Meiboom-Gill (CPMG) sequence was applied, with a τ interval of 400 μs, 80 loops, 32 data collections, an acquisition time of 1.5 s, a relaxation delay of 2 s and a fixed receiver gain, was used to suppress broad signals arising from large molecular weight serum/plasma components (e.g. albumin). In this way, the CPMG pulse sequence retains resonances from small molecular weight metabolites and mobile side chains of lipoproteins, providing an accurate measurement of these parameters in the serum/plasma sample. All serum/plasma spectral data acquisitions were therefore performed using the CPMG pulse sequence, as per standard NMR metabolomics literature (Soininen et al., 2009).

Free induction decays of the pulse sequences were zero-filled by a factor of 2 and multiplied by an exponential function corresponding to 0.30 Hertz (Hz) line broadening prior to Fourier transformation. For quality control (QC), pooled serum/plasma samples were spread throughout the run to monitor technical variation. ¹H COSY spectra were acquired on at least one sample in each disease classification to aid in metabolite identification. When required, further confirmation was achieved by 1D TOCSY or 2D TOCSY NMR experiments, or with spiking experiments with known candidate compounds. Metabolite assignments were further confirmed by referencing to literature values and the Human metabolome database (HMDB) (Wishart et al., 2018).

The buffered NMR samples were then stored at −80° C. until NMR analysis. Immediately before NMR experiments, the NMR samples were thawed at room temperature, briefly vortexed, and then transferred to a 5 mm borosilicate glass tube (Norell™ 502-7) via a glass pipette.

Global Metabolomics—CPMG Spectra Data Acquisition

All ¹H NMR experiments for global metabolomics were performed in-house at the Department of Chemistry, University of Oxford using a 700-MHz Bruker AVIII spectrometer, with the CPMG relaxation editing pulse sequence for spectra acquisition. Technical specifications of the NMR experiments and details of data handling are as described in Materials and Methods. In all, 191 metabolite ‘bins’ were available for multivariate statistical analysis.

Targeted Metabolomics—AXINON® lipoFIT® Spectra Data Acquisition

Targeted metabolomics was performed with the AXINON® lipoFIT® system at numares AG, Regensburg, Germany, using a 500-MHz Bruker NMR spectrometer with the NOESY pulse sequence for spectra acquisition. This test system deconvolutes the broad methyl lipoprotein resonance of the ¹H NMR NOESY spectrum into its constituent parts, allowing for the direct measurement and quantification of the cholesterol content, number of particles, and mean particle diameter of each lipoprotein subpopulation. Lipoprotein groups measured include VLDL, LDL, IDL, and HDL, with each group further divided into large and small subpopulations. Additionally, the AXINON® lipoFIT® system provides absolute quantification of glucose as well as metabolites located close to the ¹H NMR lipoprotein resonances, as the resonances of these metabolites overlap with those arising from lipoproteins. These metabolites include lactate, glucose, alanine, as well as the BCAA—isoleucine, leucine and valine. In all, 29 variables from targeted metabolomics were available for multivariate statistical analysis.

Serum NfL Level Determination

Serum NfL levels were measured using the Simoa® assay (Quanterix, Massachusetts, USA) performed at the University of Basel, in collaboration with Dr Jens Kuhle. Assay techniques and principles have been previously described (Disanto et al., 2017; incorporated herein be reference). All laboratory personnel were blinded to the assignment of patient groups.

NMR Spectral Data Processing

All spectra were processed in Topspin 3.6.1 (Bruker BioSpin Gmbh, Rheinstetten, Germany) in which they were phased, baseline corrected using a third degree polynomial, and chemical shifts referenced to the lactate doublet resonance centred at chemical shift 1.33 ppm, a commonly used method of spectra referencing (Lu et al., 2018; Pontes et al., 2019; Verwaest et al., 2011). The NMR spectra were then visually inspected for errors in baseline correction, referencing, spectral distortion, or contamination with exogenous products (e.g. ethanol, propylene glycol). Processed spectra were then transferred to ACD/Labs Spectrus Processor Academic Edition 12.01 (Advanced Chemistry Development, Inc., Toronto, Canada) whereby the water signal from 4.20-5.20 ppm was excluded due to baseline distortion, as were signal-free noise regions. The remaining regions of the spectra between 0.80-4.20 ppm and 5.20-8.50 ppm were split into 0.02 ppm fixed-width bins. Integral values of each individual spectral bin were obtained and normalised to the integral value of the entire spectrum (i.e. constant-sum-normalisation) such that the integral value of the whole spectrum is 1 (Worley et al., 2013). This data matrix was used for multivariate statistical analysis.

Multivariate Statistical Analysis

OPLS-DA was used to interrogate the CPMG (global metabolomics) and AXINON® lipoFIT® (targeted metabolomics) data sets to identify metabolic differences between the groups of patients as defined above. Details of the OPLS-DA approach are as described in the paragraph below.

Multivariate analysis/model building was performed in R software using the ropls package. Orthogonal partial least squares discriminant analysis (OPLS-DA) with 10-fold external cross validation and repetition was used to identify linear combinations of metabolites which distinguish between the groups of interest. Models were validated on independent test data and by permutation testing. To identify which are the most discriminatory variables driving the separation between classes, variable importance in projection (VIP) scores derived from the OPLS-DA models are computed.

Univariate Statistical Analysis

All other statistical analyses (comparative, logistic regression, receiver operating curve [ROC], correlation analyses) were performed with STATA software (Release 14, College Station, TX: Statacorp LP, USA) and GraphPad Prism (version 6, California, USA). Comparative analyses between two groups were performed using Mann-Whitney U test or two-sample t-test as appropriate for continuous variables. For comparisons between three groups, one-way ANOVA or Kruskal Wallis test were used, with pair-wise post-hoc corrections using Bonferroni/Tukey and Dunn tests respectively. Chi-square or Fisher exact tests were used for categorical variables depending on the size of the expected frequency, with Bonferroni correction when comparing ≥3 groups. Two-way ANOVA was used to compare across groups and time points/treatment, with Sidak's test for multiple comparison corrections. Pearson's or Spearman's correlation was used to explore correlations depending on data normality. Two-tailed p values of <0.05 were considered statistically significant.

In other words, univariate analysis of demographic, clinical, and metabolite data was performed using STATA software (release 14, Texas, USA) and GraphPad Prism (version 6, California, USA). Comparative analyses between patient groups were performed using one-way ANOVA or Kruskal Wallis test as appropriate for continuous variables, with pair-wise post-hoc corrections using Bonferroni and Dunn tests respectively. Chi-square or Fisher exact tests were used for categorical variables as appropriate, with Bonferroni correction when comparing ≥3 groups. Pearson's or Spearman's correlation was used to explore correlations depending on data normality. Two-tailed p values of <0.05 were considered statistically significant and data was presented as mean±SD unless stated otherwise.

### Example 1—Global Metabolomics (CPMG Spectral Data)

ln R vs. LR≥24 M Patients

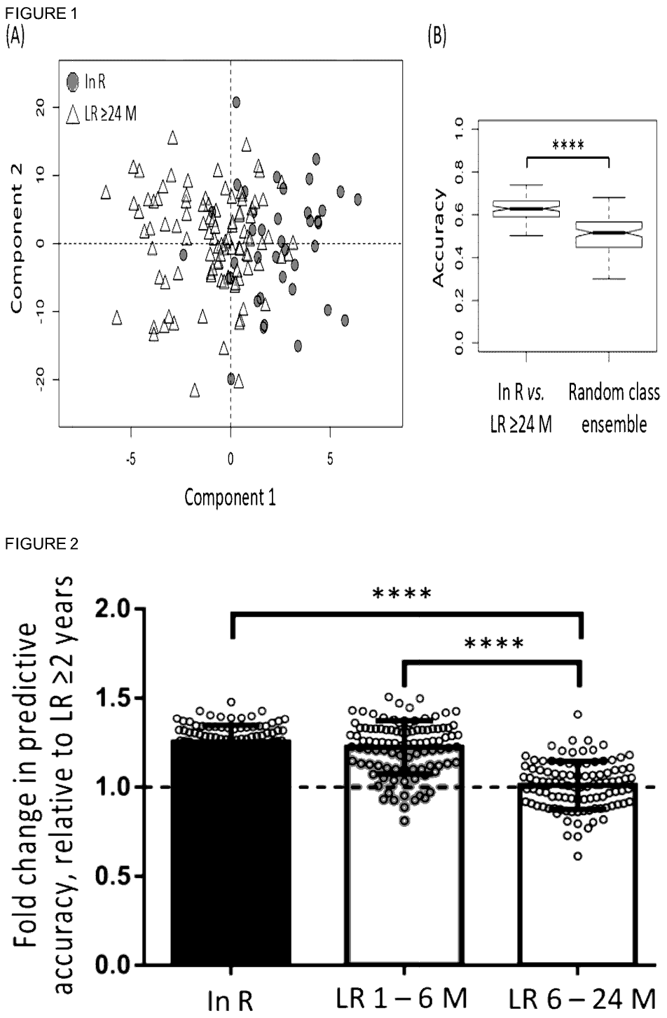

To identify global metabolic signatures and perturbations reflective of clinical relapses, OPLS-DA was used to construct discriminatory models using CPMG spectral data to distinguish between ln R and LR≥24 M patients. The representative OPLS-DA scores plot showed a moderate separation between ln R and LR≥24 M patients (FIG. 1A). The mean predictive accuracy for the ensemble of the OPLS-DA models of ln R vs. LR≥24 M patients was significantly higher than the mean predictive accuracy of the ensemble created by random class assignments (mean±SD, 62.6±4.8% vs. 50.9±8.2%; p<0.0001) (FIG. 1B), validating the metabolic differences seen between the two groups of patients.

LR 1-6 M Vs. LR≥24 M Patients, and LR 6-24 M Vs. LR≥24 M Patients

To explore how long global metabolic perturbations persist after relapses, OPLS-DA models were constructed using CPMG spectral data for LR 1-6 M as well as for LR 6-24 M patients against the reference comparator, i.e. LR≥24 M patients. The mean predictive accuracy for the ensemble of the OPLS-DA models for LR 1-6 M vs. LR≥24 M patients was significantly higher than the mean predictive accuracy of the random class ensemble (mean±SD, 61.2±7.5% vs. 48.8±8.5%; p<0.0001). In contrast, the mean accuracy of the OPLS-DA models for LR 6-24 M vs. LR≥24 M patients was not different from the mean accuracy of the random class ensemble (mean±SD, 50.5±6.7% vs. 50.6±7.8%; p=0.971).

The fold change in the predictive accuracy (normalised to the accuracy of random chance, i.e. 50%) of each patient group, using LR≥2 years patients as reference comparator, is shown in FIG. 2. Taking these findings in totality, this implies that global metabolic perturbations last for at least 6 months after a clinical relapse.

Identifying Discriminatory Metabolites from the in R Vs. LR≥24 M OPLS-DA Models

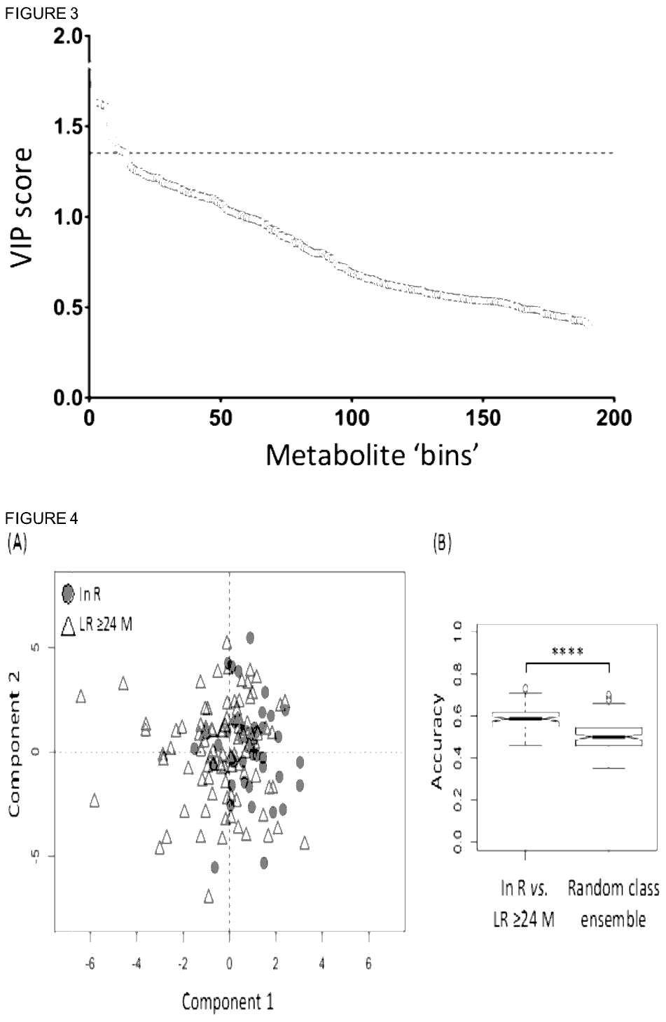

To identify the top discriminatory metabolites (and potential metabolite biomarkers of relapse) from the ln R vs. LR≥24 M OPLS-DA models, VIP scores were generated. The VIP score cut-off at 1.35 was determined by identifying a ‘drop-off’ on the VIP ranking plot (FIG. 3). Metabolites with VIP scores above this cut-off are detailed in Table 2. These consisted predominantly of lipoproteins, amino acids and glucose. As most (i.e. two thirds) of these discriminatory metabolites were detected by the AXINON® lipoFIT® system, targeted metabolomics was performed next. Although targeted metabolomics covers a smaller region of the NMR spectra, it allows for detailed lipoproteins, amino acids and glucose analyses, as well as absolute quantification of these metabolites. The additional metabolites measured by the AXINON® lipoFIT® system that were not identified to be highly discriminatory by global metabolomics included alanine, isoleucine, lactate and valine.

TABLE 2
Top discriminatory metabolites from global (CPMG) metabolomics
distinguishing In R vs. LR ≥24 M patients.

| | Chemical shift of contributing |
|---|---|
| Discriminatory metabolites | spectral ‘bins’ (VIP score, VIP rank) |
| Lipoprotein —CH3 | 0.80-0.86 ppm (1.36, 12) |
| (HDL/LDL dominated) | |
| Lipoprotein —CH3 | 0.86-0.92 ppm (1.62, 5) |
| (VLDL dominated) | |
| Leucine* | 0.94-0.98 ppm (1.36, 13); |
| | 1.62-1.78 ppm; 3.70-3.79 ppm |
| Lipoprotein —(CH₂)ₙ | 1.15-1.30 ppm |
| (HDL/LDL dominated) | |
| Lysine | 1.37-1.55 ppm; 1.65-1.75 ppm; |
| | 1.83-1.94 ppm; 3.00-3.05 ppm |
| Lipoprotein βCH₂ | 1.53-1.61 ppm (1.63, 3) |
| N-acetylated glycoprotein | 1.93-2.10 ppm |
| (e.g. NAC/═CH—CH₂—CH₂—)* | |
| Asparagine | 2.80-3.00 ppm; 3.96-4.02 ppm |
| Glucose* | 3.17-3.95 ppm; 4.63-4.66 ppm; |
| | 5.22-5.25 ppm |
| Phenylalanine | 7.32-7.44 ppm (1.60, 7); |
| | 3.1-3.3 ppm; 3.9-4.0 ppm |
| β-hydroxybutyrate | 1.19-1.21, 2.27-2.45 |
| Myo-inositol | 3.63-3.65 ppm, 3.53-3.58 ppm, |
| | 3.93-3.98 ppm, 3.25-3.29 ppm |

*indicates metabolites measured and quantified by AXINON ® lipoFIT ® system (targeted metabolomics, see Example 2). Chemical shift ranges are
reported relative to lactate —CH₃ referenced at 1.33 ppm.

### Example 2—Targeted Metabolomics (AXINON® lipoFIT® Spectral Data)

ln R Vs. LR≥24 M Patients

To identify if there were metabolomics perturbations in relapses using a targeted metabolomics approach, OPLS-DA was employed to interrogate the AXINON® lipoFIT® parameters obtained from ln R and LR≥24 M patients. The mean predictive accuracy for the ensemble of the OPLS-DA models of ln R vs. LR≥24 M patients was significantly higher than the mean predictive accuracy of the ensemble created by random class assignments (mean±SD, 58.1±5.5% vs. 50.5±7.0%; p<0.0001) (FIG. 4), confirming the presence of targeted metabolic differences between the two groups of patients. The decrease in predictive accuracy as well as the less obvious separation of the patient groups on the OPLS-DA scores plot is likely due to lesser amount of data available within a targeted region of the NMR spectra, as compared to the OPLS-DA models generated by global metabolomics.

Identifying Discriminatory Metabolites from the in R Vs. LR≥24 M OPLS-DA Model

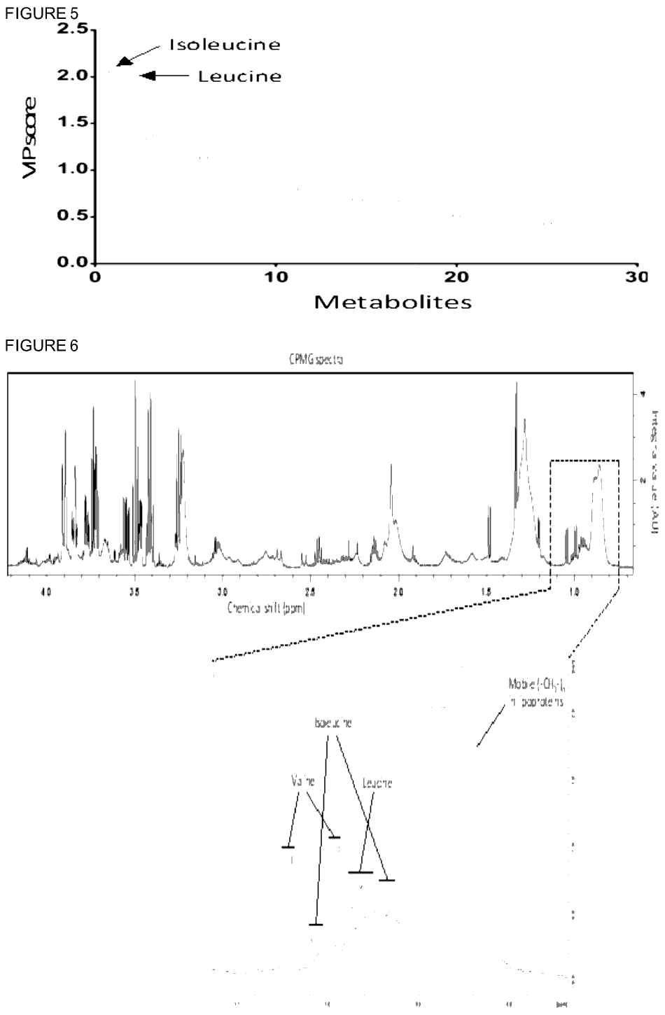

VIP scores from the ln R and LR≥24 M OPLS-DA models were generated to elucidate the principal discriminatory metabolites from targeted metabolomics. The VIP ranking plot revealed isoleucine and leucine (both are BCAA) as the two most important metabolites (FIG. 5), with VIP scores of 2.05 and 2.01 respectively. BCAA resonances are shown in FIG. 6.

Isoleucine was identified as a top discriminatory metabolite in targeted metabolomics. Of note, the top lipoprotein parameter from the AXINON® lipoFIT® was LDL-s (mean diameter of LDL particles) which was ranked third based on its VIP score of 1.35.

Outcome of Examples 1 and 2

A principle outcome of Examples 1 and 2 is that the following metabolites have been identified a biomarkers (e.g. based on significant concentration change ln R vs. LR≥24 M) for confirming that MS patient is suffering a relapse:

TABLE 3

| Metabolite | Fold change In R vs. LR ≥24 M % | | Chemical Shift (PPM) |
|---|---|---|---|
| Leucine | −4.3 | (e.g. decreased in relapse) | 0.94-0.98; 1.62-1.78; |
| | | | 3.70-3.79 |
| Lysine | 5.2 | (e.g. increased in relapse) | 1.37-1.55; 1.65-1.75; |
| | | | 1.83-1.94; 3.00-3.05 |
| Asparagine | 5.9 | (e.g. increased in relapse) | 2.80-3.00; 3.96-4.02 |
| Phenylalanine | −6.5 | (e.g. decreased in relapse) | 7.32-7.44; 3.1-3.3; 3.9-4.0 |
| Glucose | 4.2 | (e.g. increased in relapse) | 3.17-3.95; 4.63-4.66; |
| | | | 5.22-5.25 |
| β-hydroxybutyrate | −5.2 | (e.g. decreased in relapse) | 1.19-1.21 ppm, 2.27-2.45 ppm |
| Myo-inositol | −4.9 | (e.g. decreased in relapse) | 3.63-3.65 ppm, 3.53-3.58 ppm, |
| | | | 3.93-3.98 ppm, 3.25-3.29 ppm |
| Lipoprotein —CH3 | −6.4 | (e.g. decreased in relapse) | 0.80-0.86 |
| (HDL/LDL dominated) | | | |
| Lipoprotein —CH3 (VLDL | −7.5 | (e.g. decreased in relapse) | 0.86-0.92 |
| dominated) | | | |
| Lipoprotein —(CH₂)ₙ | −8.1 | (e.g. decreased in relapse) | 1.15-1.30 |
| (HDL/LDL dominated) | | | |
| Lipoprotein βCH₂ | 3.8 | (e.g. increased in relapse) | 1.53-1.61 |
| N-acetylated glycoprotein | −4.5 | (e.g. decreased in relapse) | 1.93-2.10 |
| (e.g. NAC1/═CH—CH2—CH2—) | | | |
| Isoleucine | −5.1 | (e.g. decreased in relapse) | 0.92-0.97; 1.00-1.03; |
| | | | 1.22-1.28; 1.43-1.51; |
| | | | 1.94-2.01; 3.65-3.68 |

### Example 3—Further Exploration of Metabolite Biomarkers of Clinical Relapses

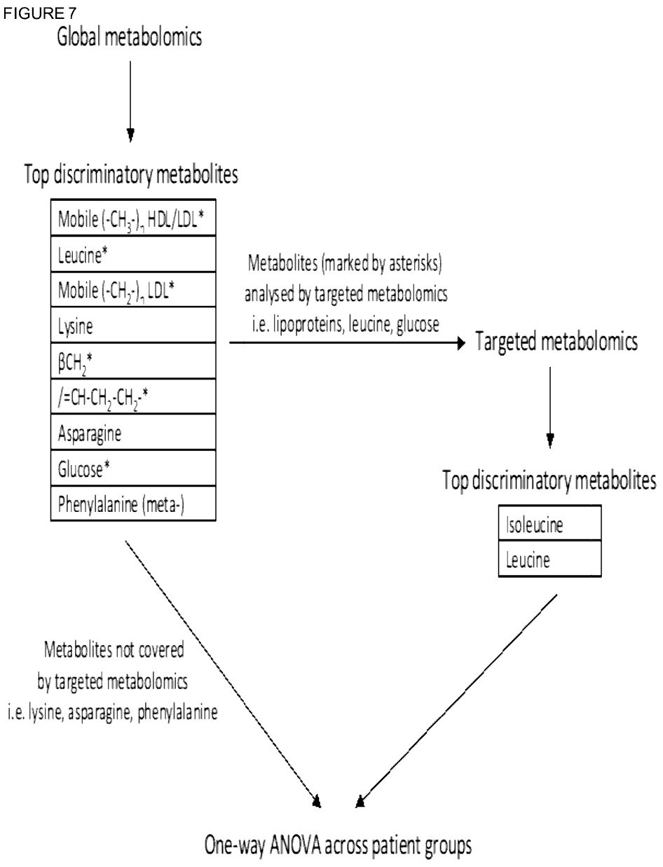

As demonstrated above, using the OPLS-DA analytical approach, global metabolic perturbations can be observed during relapses and up to six months after relapses. The top discriminatory metabolites from targeted (isoleucine and leucine) and global metabolomics (metabolites listed in Table 2 not marked by an asterisk) distinguishing ln R vs. LR≥24 M patients were shortlisted for further demonstration of the associated of metabolite biomarkers with clinical relapses. One-way ANOVA was performed for each of these shortlisted metabolite across the four patient groups, using LR≥24 M patients as the reference group. The workflow for choosing the metabolites for ANOVA is illustrated in FIG. 7. For consistency, discriminatory metabolomics from global metabolomics (i.e. lipoproteins, leucine and glucose) that are already covered in-depth by targeted metabolomics (AXINON® lipoFIT® system) were not included in this analysis.

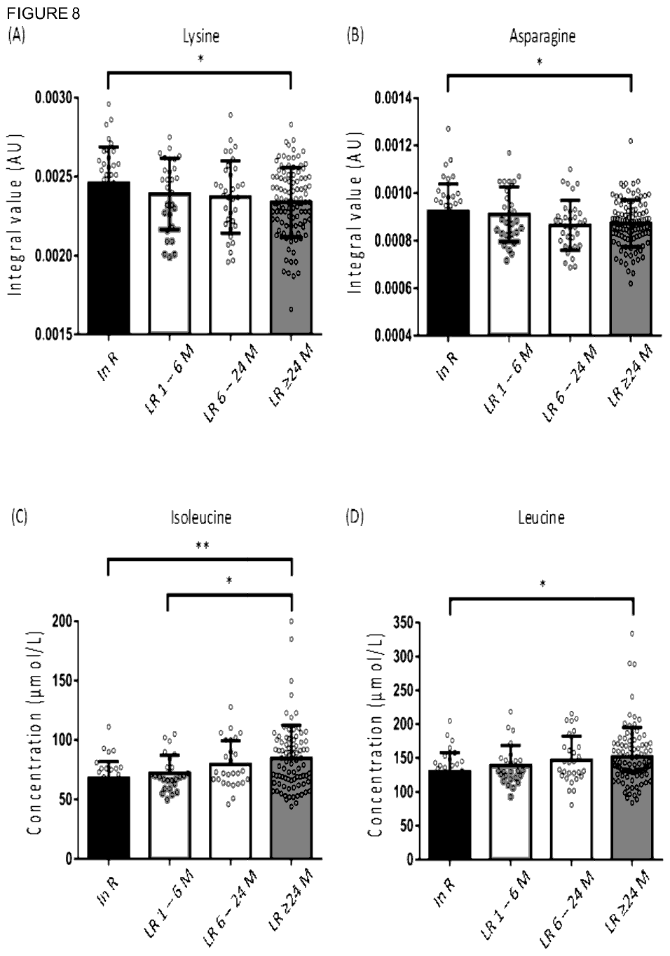

Lysine and asparagine (from global metabolomics), as well as isoleucine and leucine (from targeted metabolomics) were significant on one-way ANOVA. Both lysine and asparagine were higher in ln R vs. LR≥24 M patients, and showed a decreasing trend over time (FIGS. 8A and B). The converse was observed for isoleucine and leucine: lower levels in ln R compared to LR≥24 M patients and increasing with time away from relapse (FIGS. 8C and D). Taking these observations in totality, this demonstrates that lysine, asparagine, isoleucine and leucine are particularly advantageous metabolite biomarkers of clinical relapses.

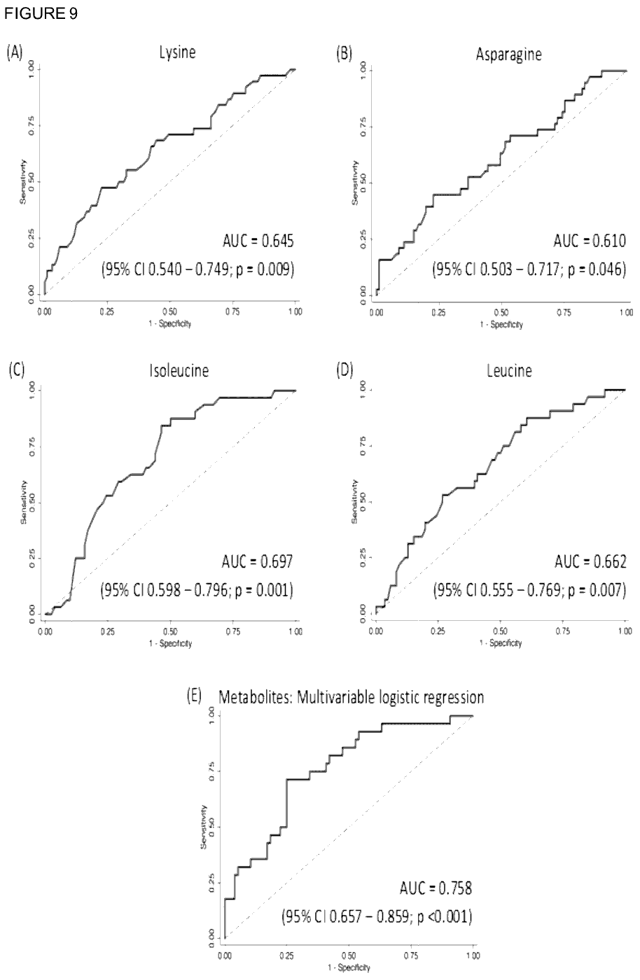

On ROC analysis to distinguish between ln R and LR≥24 M patients, the univariate AUC of the four metabolite biomarkers ranged from 0.610 to 0.697 (FIG. 9A-D). Next, to explore if the combination of the four metabolites could enable higher discriminatory ability, the four metabolites were used as independent variables in a multivariable logistic regression model followed by ROC analysis. This resulted in an improved AUC of 0.758 (FIG. 9E).

### Example 4—Exploring Serum NfL as a Potential Biomarker of Clinical Relapses

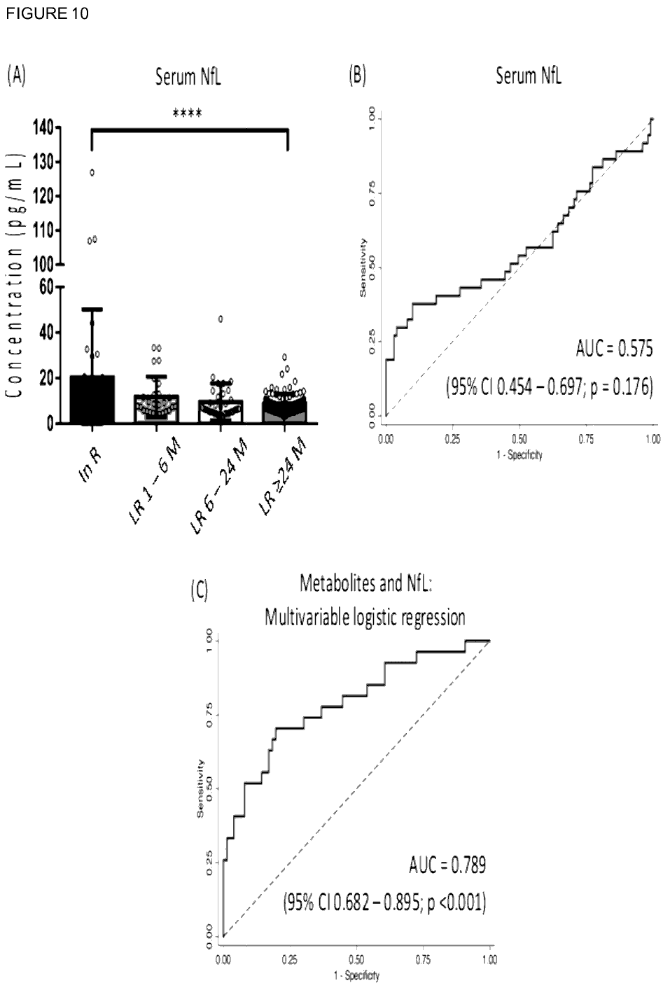

The previous sections demonstrated that using both global and targeted metabolomics approaches, four metabolites in particular examined further to demonstrate they serve as biomarkers of relapse. Serum NfL has been suggested to be a potential biomarker to inform on MS inflammatory activity and is reported to be elevated in clinical relapses, thus its diagnostic performance in this cohort of patients was explored. One-way ANOVA of serum NfL levels, using LR≥24 M patients as the reference group, showed that ln R patients had higher levels of serum NfL compared to LR≥24 M patients, although this appeared to be driven by outliers with very elevated levels of serum NfL (FIG. 10A). Indeed, when the three highest serum NfL values (i.e. outliers) from the ln R group were removed, the one-way ANOVA was no longer statistically significant (p=0.054) and the post-hoc Holm-Sidak's test comparing ln R vs. LR≥24 M was also not statistically significant (p=0.061). ROC analysis of all data points showed an AUC of 0.575 for serum NfL in distinguishing between ln R vs. LR≥24 M patients (FIG. 10B).

That being said, it was next explored if the ability to distinguish ln R and LR≥24 M patients could be increased by combining 4 metabolites and NfL. A multivariable logistic regression model was constructed followed ROC analysis (FIG. 10C), showing a slightly increased AUC of 0.789, as compared to using the combination of the four metabolites alone.

### Example 5—Metabolites as Individualised, Responsive Biomarkers of Relapses

From the previous sections, lysine, asparagine, isoleucine and leucine were identified to be advantageous metabolite biomarkers of relapses. It was next explored whether these metabolites would be: (1) applicable in an individualised manner (i.e. within an individual patient), and (2) be responsive enough such that the change in its levels between diseased states must be observable within a clinically useful time frame.

To this end, nine patients (under the MET cohort collection) who had paired relapse-remission samples (i.e. relapse first followed by remission), and with the remission sample collected within 6 months of relapse onset, were identified. The clinical characteristics of these nine patients are shown in Table 4. Of note, none of these patients received steroids at the time of relapse blood sampling.

TABLE 4
Clinical characteristics of the nine patients with paired relapse-
remission samples, with data collected at the relapse time point.

| | Patients with paired | |
|---|---|---|
| | relapse-remission | |
| | (n = 9) | |
| Age in years, mean (SD) | 40.2 | (7.1) |
| Female, No. (%) | 6 | (66.7) |
| White, No. (%) | 9 | (100.0) |
| Recent/current steroid use, No. (%) | 0 | (0.0) |
| DMT use, No (%) | 4 | (44.4) |
| Alemtuzumab | 1 | (25.0) |
| Glatiramer acetate | 2 | (50.0) |
| Interferons | 1 | (25.0) |
| EDSS, median (range) | 4.0 | (2.0-6.5)* |
| Disease duration in years, median (range) | 14.8 | (1.9-23.5) |
| No comorbidities, No. (%) | 4 | (44.4) |
| BMI, median (range) | 30.3 | (24.2-46.4) |
| Interval between relapse onset and | 3.6 | (2.3-5.6) |
| remission sampling in months, median | | |
| (range) | | |
| Presence of new T₂ lesion/s within | Not known as none had | |
| last 1 year, referenced to baseline | earlier baseline reference | |
| scan done ≤1 year apart, No. (%) | scan done ≤1 year | |
| Presence of GAD enhancing lesion/s | 1/5 | (20) |
| within last 1 year, No. (%) | | |
| Presence of new T₂ lesion/s within | 2/4 | (50) |
| next 6 months, referenced to baseline | | |
| scan ≤1 year apart, No. (%) | | |
| Presence of GAD enhancing lesion/s | 3/6 | (50) |
| within next 6 months, No. (%) | | |

*Median EDSS at remission sampling was 2.5 (range 1.0-6.5).

Univariate Analysis

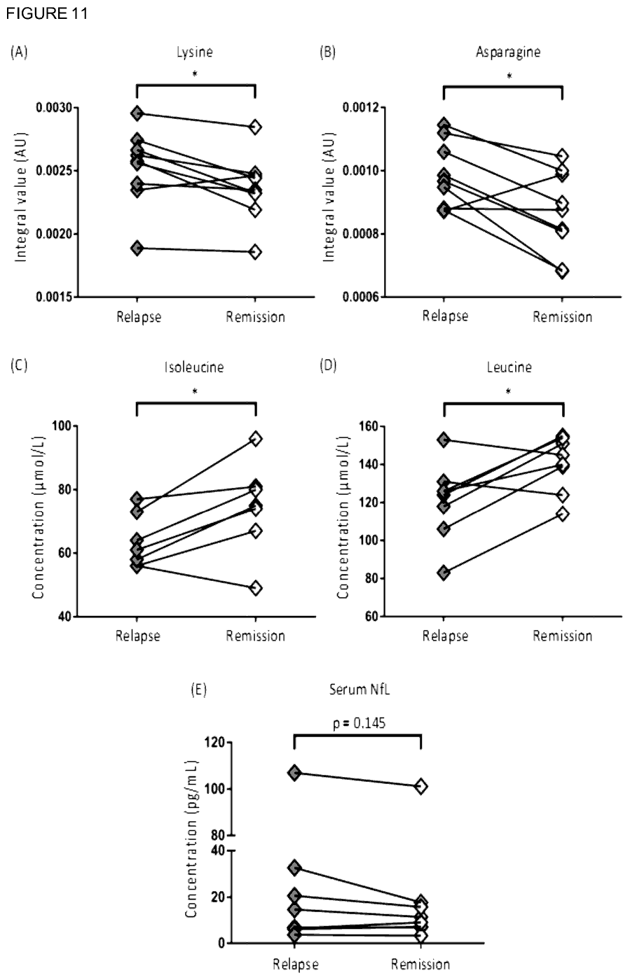

On paired t-test, all four metabolites showed significant differences in their levels during relapse and in remission (FIGS. 11A-D), and the direction of change was consistent with that observed when comparing at group level across the four groups of patients (FIGS. 8A-D). There was no difference in serum NfL levels within this 6-month time frame (FIG. 11E). Six out of the eight patients (one patient had missing data for the relapse sample and was excluded from analysis) had lower serum NfL levels during remission while two had higher levels. It was observed that the change in serum NfL levels was gradual and there were considerable inter-individual differences in NfL levels during relapses, consistent with observations from group comparisons (FIG. 8).

Multivariate Analysis

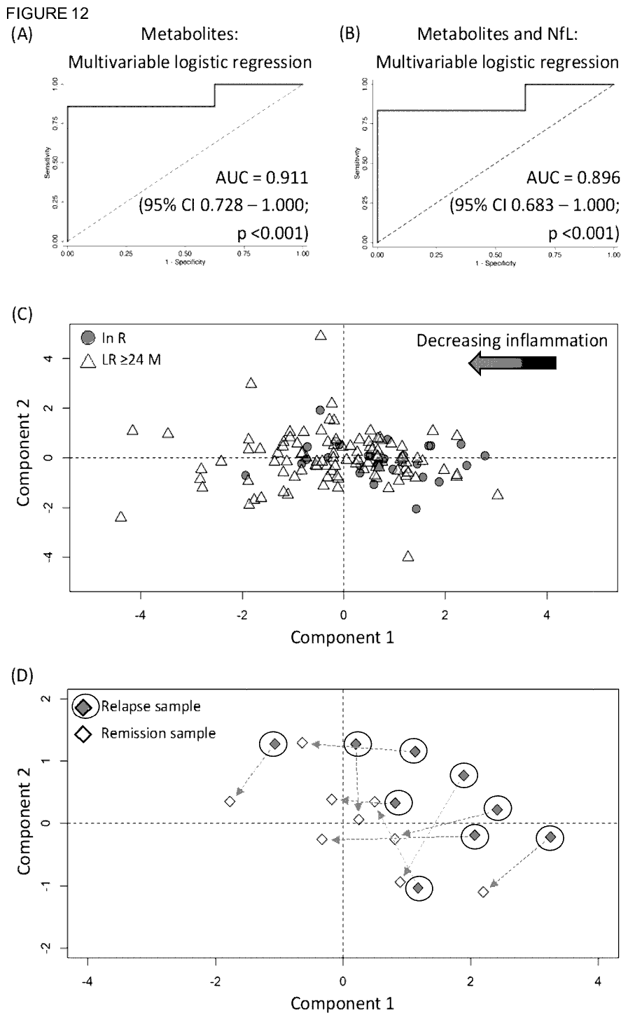

The majority of these patients had complete concordance in the direction of change for all four metabolites. Given these observations, multivariate approaches were employed to determine if a combination of these four metabolites (i.e. a metabolic constellation), rather than individual metabolites, could be used as a composite biomarker. Using the four metabolites as independent variables in a multivariable logistic regression approach followed by ROC analysis, an AUC of 0.911 was achieved (FIG. 12A). The addition of serum NfL into this multivariable regression model resulted in slight decrease in AUC to 0.896 (FIG. 12B).

To further extend this demonstration of a composite metabolic biomarker, a vector-based OPLS-DA approach was employed. A representative OPLS-DA scores plot (generated using 7-fold internal cross-validation) of ln R vs. LR≥24 M patients was first constructed (FIG. 12C) using the same data used to generate the scores plot in FIG. 1A, however excluding the relapse data of the nine patients who had paired relapse-remission samples. This was done to allow the data (from these nine patients) to be used as an independent predictive set. The x-axis of the OPLS-DA scores plot (FIG. 12C), i.e. along the first component, can be thought to represent inflammation, with a leftward vector signifying decreasing inflammatory activity. Data from the paired relapse-remission samples were then inserted into this OPLS-DA scores plot as a predictive set (FIG. 12D). All remission samples, except one, moved in a leftward direction (along the first component) with respect to their paired relapse sample, consistent with the vector representing decreasing inflammation.

### Example 6—Demonstrating Utilisation of Metabolites as Predictive Biomarkers of Relapses

Isoleucine and leucine were chosen for further analysis to explore whether the biomarkers find utility in predicting onset of a relapse.

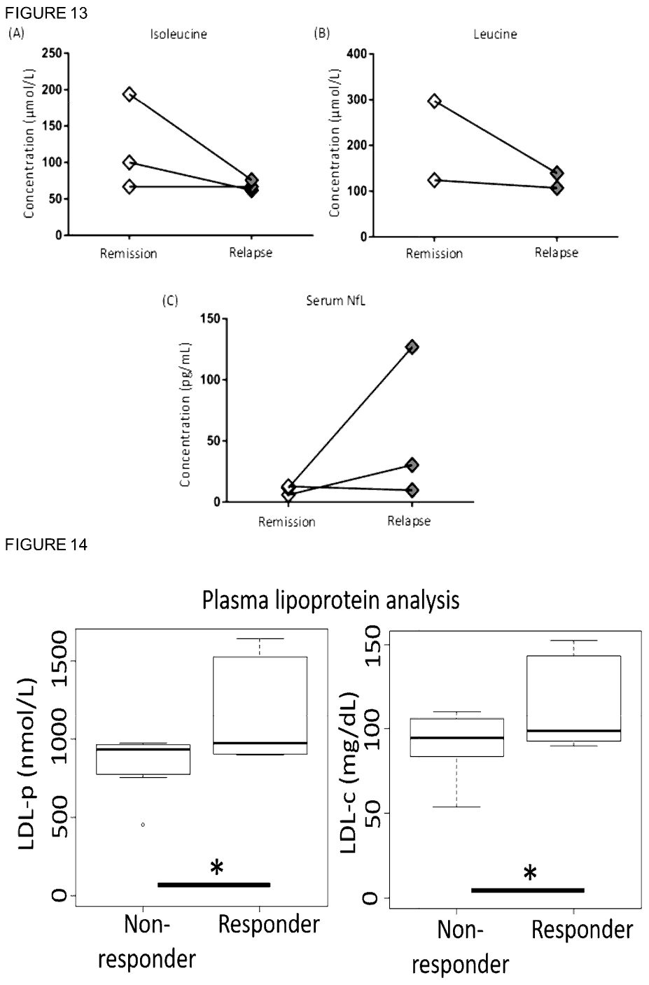

To explore if the identified metabolites can be predictive biomarkers of relapses, three patients (from the MET cohort) who had remission samples prior to relapse samples were identified. The mean interval between remission and relapse was 14.1 months. Two of the three patients had higher isoleucine levels during remission compared to in relapse, while the levels remained the same for one patient (FIG. 13A). For leucine, two patients showed higher levels during remission (FIG. 13B). The leucine measurement in remission for one patient was not available and was thus excluded from analysis. The direction of change was consistent with that described in the previous sections. For serum NfL, most patients showed lower levels in remission (FIG. 13C). This observation could mean that isoleucine and leucine are reflective of the state of immune activation.

### Example 7—Addressing Potential Confounders for the Identified Metabolite Biomarkers

As illustrated in Table 1, several baseline characteristics were different across the four patient groups; notably age, steroid use, and DMT use were dissimilar between ln R vs. LR≥24 M patients on post-hoc analysis. Two approaches were used to address these potential confounders: (1) for quantitative variables, correlation of the potential confounding variable with each of the four identified metabolite biomarkers was performed, and (2) for categorical variables, the levels of the four metabolites were explored stratified by the potential confounding variable (i.e. steroid use and DMT use).

There were no correlations between any of the four metabolite biomarkers with age, EDSS and disease duration (the highest R² was 0.113 between age and lysine) across the entire cohort of patients (n=201). There were no differences in any of the metabolite concentrations stratified by steroid use in the whole cohort (7 steroid users, 194 non-steroid users) and indeed within ln R patients (5 steroid users, 33 non-steroid users). For DMT use, there were higher isoleucine levels in DMT users (mean±SD, 82.3±25.6 μmol/L vs. 72.8±18.7 μmol/L; p=0.011) across the entire cohort (125 DMT users, 76 non-DMT users). However, no differences in isoleucine levels were observed between DMT users and non-users within ln R patients (18 DMT users, 20 non-DMT users) (p=0.120) as well as within LR≥24 M patients (75 DMT users, 26 non-DMT users) (p=0.304). There were also no significant associations/correlations of the discriminatory metabolites with smoking status, alcohol intake, and time from last meal.

### Example 8—Association of Metabolite Biomarkers with MRI Indices of Inflammatory Activity

The association of certain identified metabolite biomarkers with MRI indices of inflammation, i.e. the presence of new T₂ lesion/s (referenced to baseline scan done 51 year apart) as well as the presence of GAD-enhancing lesion/s within the last 1 year, were explored. Interestingly, lysine (mean±SD, 25.5×10⁻⁴±2.1×10⁻⁴ AU vs. 23.7×10⁻⁴±2.4×10⁻⁴ AU; p=0.008) and asparagine (mean±SD, 9.6×10⁻⁴±1.2×10⁻⁴ AU vs. 8.9×10⁻⁴±1.2×10⁻⁴ AU; p=0.048) levels were significantly higher in patients who had GAD-enhancing lesions.

### Example 9—Demonstrating Treatment Response Via Metabolomics

It was explored whether metabolomics analysis can be used to monitor a patient's response to a treatment, e.g. to distinguish a responder from a non-responder. Associated data are provided in FIGS. 14-16.

FIG. 14 shows results of analysis of plasma lipoprotein subclass particle number (-p), size, and cholesterol content (-c) of non-responders (defined as clinical relapse during 6 month follow-up) and responders (defined as no clinical relapse during follow-up) to first-line MS therapy using the AXINON® lipoFIT® platform. A significant increase in LDL particle number and cholesterol content was observed in the responder group relative to the non-responders.

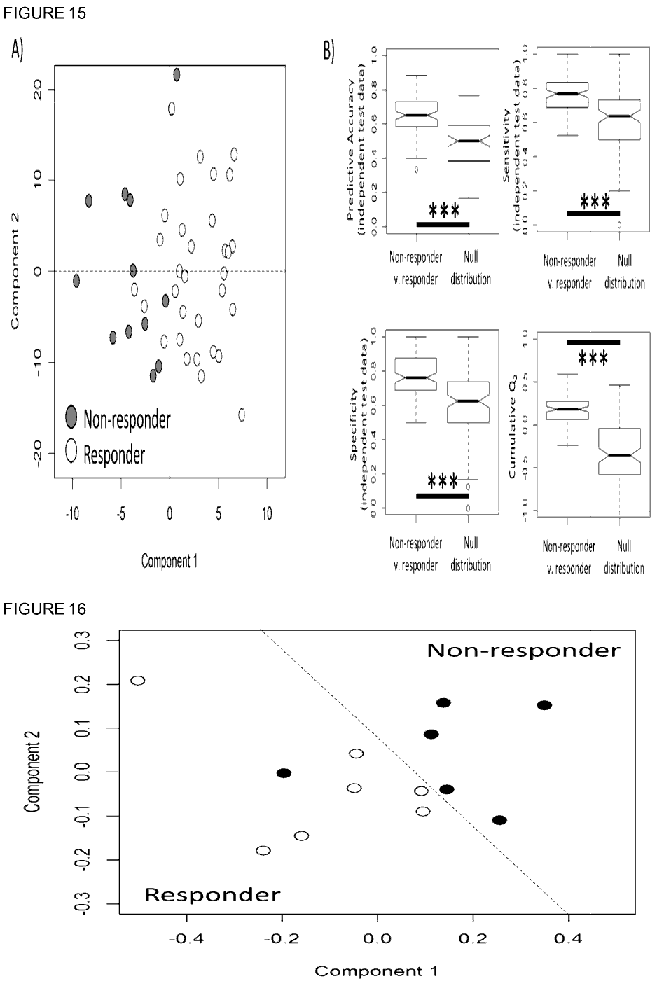

FIG. 15 shows results of OPLS-DA NMR metabolomics analysis of plasma samples from a cohort of 44 people with MS receiving glatiramer acetate treatment provides additional evidence that plasma metabolites can distinguish between treatment responders (defined as no clinical relapse during 6 month follow up) and non-responders (defined as clinical relapse during 6 month follow up). A) A representative OPLS-DA scores plot (from the ensemble of 1000 cross-validated models generated) illustrates excellent discrimination between classes. B) A subset of the model validation metrics are provided including the predictive accuracy, sensitivity, specificity and cumulative Q₂ to illustrate statistical significance of the model, robustness, and validation on independent data. Each box plot presents 1000 predictive values determined on randomly selected test sets. A permutation test generates the null distribution (1000 models) in order to validate the model and confirm discrimination has not occurred by chance. Kolmogorov-Smirnov test p-values<0.001 are represented by ***.

FIG. 16 shows results of unsupervised analysis of baseline blood samples from people with MS who go on to respond/not respond to 1st line therapy spontaneously clusters. This pilot data illustrates that significant differences in the blood metabolite profile exist which are predictive of response to 1st line treatments.

Discussion of Examples

The above Examples demonstrate that: (1) metabolic perturbations are present in patients during relapses using both global and targeted metabolomics, as compared to patients with no relapses for the past two years, (2) in particular, four discriminatory metabolites that were significant on ANOVA (lysine, asparagine, isoleucine and leucine) across the different patient groups showed a consistent trend (either increasing or decreasing) with time away from relapse, and advantageously, (3) these metabolites are informative in an individualised manner within a clinically useful time frame. Taking these observations in totality, this demonstrates that the identified metabolites are advantageous biomarkers of clinical relapses.

Without wishing to be bound by theory, it is believed the metabolic perturbation seen in MS relapses is likely to be due to the summative effects of various immunopathological processes that occur during these inflammatory events: (1) activation of peripheral T cells and monocytes, and their subsequent access into the CNS, (2) activation of resident microglial and astrocytes, (3) initiation of injurious effector mechanisms leading to the production of ROS and mitochondrial stress, and (4) demyelination with possible axonal injury. Advantageously, while many/most of these pathophysiological processes may occur within the CNS, the metabolic perturbations can be detected in alternative samples (e.g. alternatively/additionally to cerebrospinal fluid), such as serum. Two possible explanations can account for this observation: (1) CNS metabolites involved in these pathophysiological processes can cross the blood brain barrier, BBB (and indeed in the opposite direction) facilitated by increased permeability on a background of an inflamed BBB, and/or (2) the metabolic perturbation is contributed mostly by peripheral processes, namely the activation of peripheral immune cells and the peripheral response to CNS injury which is mediated primarily by the liver. The postulated roles of the identified metabolite biomarkers in these processes will now be discussed.

### REFERENCES

Bervoets, L., E. Louis, G. Reekmans, L. Mesotten, M. Thomeer, P. Adriaensens and L. Linsen (2015). “Influence of preanalytical sampling conditions on the 1H NMR metabolic profile of human blood plasma and introduction of the Standard PREanalytical Code used in biobanking.” Metabolomics 11(5): 1197-1207.
Disanto, G., C. Barro, P. Benkert, Y. Naegelin, S. Schadelin, A. Giardiello, C. Zecca, K. Blennow, H. Zetterberg, D. Leppert, L. Kappos, C. Gobbi, J. Kuhle and G. Swiss Multiple Sclerosis Cohort Study (2017). “Serum Neurofilament light: A biomarker of neuronal damage in multiple sclerosis.” Ann Neurol 81(6): 857-870.
Lu, Y., X. Zhang, L. Zhao, C. Yang, L. Pan, C. Li, K. Liu, G. Bai, H. Gao and Z. Yan (2018). “Metabolic Disturbances in the Striatum and Substantia Nigra in the Onset and Progression of MPTP-Induced Parkinsonism Model.” Front Neurosci 12: 90.
Pontes, T. A., A. D. Barbosa, R. D. Silva, M. R. Melo-Junior and R. O. Silva (2019). “Osteopenia-osteoporosis discrimination in postmenopausal women by 1H NMR-based metabonomics.” PLoS One 14(5): e0217348.
Soininen, P., A. J. Kangas, P. Wurtz, T. Tukiainen, T. Tynkkynen, R. Laatikainen, M. R. Jarvelin, M. Kahonen, T. Lehtimaki, J. Viikari, O. T. Raitakari, M. J. Savolainen and M. Ala-Korpela (2009). “High-throughput serum NMR metabonomics for cost-effective holistic studies on systemic metabolism.” Analyst 134(9): 1781-1785.
Verwaest, K. A., T. N. Vu, K. Laukens, L. E. Clemens, H. P. Nguyen, B. Van Gasse, J. C. Martins, A. Van Der Linden and R. Dommisse (2011). “(1)H NMR based metabolomics of CSF and blood serum: a metabolic profile for a transgenic rat model of Huntington disease.” Biochim Biophys Acta 1812(11): 1371-1379.
Wishart, D. S., Y. D. Feunang, A. Marcu, A. C. Guo, K. Liang, R. Vazquez-Fresno, T. Sajed, D. Johnson, C. Li, N. Karu, Z. Sayeeda, E. Lo, N. Assempour, M. Berjanskii, S. Singhal, D. Arndt, Y. Liang, H. Badran, J. Grant, A. Serra-Cayuela, Y. Liu, R. Mandal, V. Neveu, A. Pon, C. Knox, M. Wilson, C. Manach and A. Scalbert (2018). “HMDB 4.0: the human metabolome database for 2018.” Nucleic Acids Res 46(D1): D608-D617.
Worley, B. and R. Powers (2013). “Multivariate Analysis in Metabolomics.” Curr Metabolomics 1(1): 92-107.
Yin, P., R. Lehmann and G. Xu (2015). “Effects of pre-analytical processes on blood samples used in metabolomics studies.” Anal Bioanal Chem 407(17): 4879-4892.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for confirming that a multiple sclerosis (MS) patient is suffering from a relapse, or for determining prognosis of a relapse in a MS patient, or for monitoring a MS patient's response to therapy, the method comprising:
a. comparing a concentration of one or more metabolite(s) present in a sample obtained from the patient with the concentration of the same one or more metabolite(s) in a reference standard, wherein the one or more metabolite(s) are selected from: lysine, leucine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, a lipoprotein having a βCH2 group, and an N-acetylated glycoprotein; or
b. comparing an intensity of one or more chemical shift region(s) of a ¹H-NMR spectrum of a sample obtained from the patient with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
c. (i) confirming or not confirming that the patient is suffering a relapse based on the comparison, or (ii) determining or not determining that the patient's prognosis is poor based on the comparison or (iii) determining or not determining that the patient is responsive to the therapy based on the comparison.

2. The method according to claim 1, wherein the method is for confirming that a multiple sclerosis (MS) patient is suffering from a relapse, the method comprising:
A. comparing a concentration of one or more metabolite(s) present in a sample obtained from the patient with the concentration of the same one or more metabolite(s) in a reference standard, wherein the one or more metabolite(s) are selected from: lysine, leucine, asparagine, phenylalanine, glucose, β-hydroxybutyrate, myo-inositol, a lipoprotein having a —CH3 group of an HDL and/or LDL, a lipoprotein having a —CH3 group of a VLDL, a lipoprotein having a —(CH2)n group of an HDL and/or LDL, a lipoprotein having a βCH2 group, and an N-acetylated glycoprotein, and
a. confirming that the patient is suffering from a relapse when:
i. the concentration of one or more metabolite(s) selected from: lysine, asparagine, glucose, and a lipoprotein having a βCH₂ group is higher in the sample relative to the reference standard, wherein the reference standard is a non-relapse reference standard; and/or
ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is lower in the sample relative to the reference standard, wherein the reference standard is a non-relapse reference standard; and/or
iii. the concentration of one or more metabolite(s) selected from: lysine, asparagine, glucose, and a lipoprotein having a βCH₂ group is the same or higher in the sample relative to the reference standard, wherein the reference standard is a relapse reference standard; and/or
iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is the same or lower in the sample relative to the reference standard, wherein the reference standard is a relapse reference standard; or
b. not confirming that the patient is suffering from a relapse, or confirming that the patient is not suffering from a relapse, when:
i. the concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is the same or higher in the sample relative to the reference standard, wherein the reference standard is a non-relapse reference standard; and/or
iii. the concentration of one or more metabolite(s) selected from: lysine, asparagine, glucose, and a lipoprotein having a βCH₂ group is lower in the sample relative to the reference standard, wherein the reference standard is a relapse reference standard; and/or
iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is higher in the sample relative to the reference standard, wherein the reference standard is a relapse reference standard; or
B. comparing an intensity of one or more chemical shift region(s) of a ¹H-NMR spectrum of a sample obtained from a patient with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
a. confirming the patient is suffering a relapse when:
i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard; and/or
ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is lower in the sample relative to the reference standard, wherein the reference standard is a non-relapse reference standard; and/or
iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard; and/or
iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard; or
b. not confirming that the patient is suffering from a relapse, or confirming that the patient is not suffering from a relapse, when:
i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard; and/or
ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard; and/or
iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard; and/or
iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a relapse reference standard.

3. The method according to claim 1, wherein the method is for determining prognosis of a relapse in a multiple sclerosis (MS) patient (preferably wherein the MS patient is in remission), the method comprising:
a. comparing a concentration of one or more metabolite(s) present in a sample obtained from the patient with the concentration of the same one or more metabolite(s) in a reference standard, wherein the one or more metabolite(s) are selected from: lysine, leucine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, a lipoprotein having a βCH2 group, and an N-acetylated glycoprotein; and
b. determining that the patient's prognosis is poor when:
i. the concentration of one or more metabolite(s) selected from: lysine, asparagine, glucose, and a lipoprotein having a βCH₂ group is higher in the sample relative to the reference standard, wherein the reference standard is a non-relapse reference standard; and/or
ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is lower in the sample relative to the reference standard, wherein the reference standard is a non-relapse reference standard; and/or
iii. the concentration of one or more metabolite(s) selected from: lysine, asparagine, glucose, and a lipoprotein having a βCH₂ group is the same or higher in the sample relative to the reference standard, wherein the reference standard is a relapse reference standard; and/or
iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is the same or lower in the sample relative to the reference standard, wherein the reference standard is a relapse reference standard; or
c. not determining that the patient's prognosis is poor, or determining that the patient's prognosis is good, when:
i. the concentration of one or more metabolite(s) selected from: lysine, asparagine, glucose, and a lipoprotein having a βCH₂ group is the same or lower in the sample relative to the reference standard, wherein the reference standard is a non-relapse reference standard; and/or
ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is the same or higher in the sample relative to the reference standard, wherein the reference standard is a non-relapse reference standard; and/or
iii. the concentration of one or more metabolite(s) selected from: lysine, asparagine, glucose, and a lipoprotein having a βCH₂ group is lower in the sample relative to the reference standard, wherein the reference standard is a relapse reference standard; and/or
iv. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is higher in the sample relative to the reference standard, wherein the reference standard is a relapse reference standard.

4. The method according to claim 1, wherein the method is for determining prognosis of a relapse in a multiple sclerosis (MS) patient (preferably wherein the MS patient is in remission), the method comprising:
a. comparing an intensity of one or more chemical shift region(s) of a ¹H-NMR spectrum of a sample obtained from a patient with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
b. determining the patient's prognosis is poor when:
i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is higher in the sample relative to the reference standard, wherein the reference standard is a non-relapse reference standard; and/or
ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is lower in the sample relative to the reference standard, wherein the reference standard is a non-relapse reference standard; and/or
iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a relapse reference standard; and/or
iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a relapse reference standard; or
c. not determining that the patient's prognosis is poor, or determining that the patient's prognosis is good, when:
i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or lower in the sample relative to the reference standard, wherein the reference standard is a non-relapse reference standard; and/or
ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or higher in the sample relative to the reference standard, wherein the reference standard is a non-relapse reference standard; and/or
iii. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a relapse reference standard; and/or
iv. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a relapse reference standard.

5. The method according to claim 1, wherein the method is for monitoring a multiple sclerosis (MS) patient's response to therapy, wherein the patient is suffering or suspected of suffering from a relapse, or is at risk of suffering a relapse, the method comprising:
a. providing a sample obtained from the patient, wherein the patient has received therapy for a relapse;
b. comparing a concentration of one or more metabolite(s) present in the sample obtained from the patient with the concentration of the same one or more metabolite(s) in a reference standard, wherein the one or more metabolite(s) are selected from: lysine, leucine, asparagine, phenylalanine, glucose, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, a lipoprotein having a βCH2 group, and an N-acetylated glycoprotein; and
c. determining that the patient is responsive to the therapy when:
i. the concentration of one or more metabolite(s) selected from: lysine, asparagine, glucose, and a lipoprotein having a βCH₂ group is lower in the sample relative to the reference standard, wherein the reference standard is a reference standard that is representative of the concentration of said one or more metabolite(s) in a sample obtained from the patient pre-administration of the therapy; and/or
ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is higher in the sample relative to the reference standard, wherein the reference standard is a reference standard that is representative of the concentration of said one or more metabolite(s) in a sample obtained from the patient pre-administration of the therapy; or
d. not determining that the patient is responsive to the therapy, or determining that the patient is not responsive to the therapy when:
i. the concentration of one or more metabolite(s) selected from: lysine, asparagine, glucose, and a lipoprotein having a βCH₂ group is the same or higher in the sample relative to the reference standard, wherein the reference standard is a reference standard that is representative of the concentration of said one or more metabolite(s) in a sample obtained from the patient pre-administration of the therapy; and/or
ii. the concentration of one or more metabolite(s) selected from: leucine, phenylalanine, β-hydroxybutyrate, myo-inositol, a lipoprotein having a —CH₃ group of an HDL and/or LDL, a lipoprotein having a —CH₃ group of a VLDL, a lipoprotein having a —(CH₂)ₙ group of an HDL and/or LDL, and an N-acetylated glycoprotein is the same or lower in the sample relative to the reference standard, wherein the reference standard is a reference standard that is representative of the concentration of said one or more metabolite(s) in a sample obtained from the patient pre-administration of the therapy.

6. The method according to claim 1, wherein the method is for monitoring a multiple sclerosis (MS) patient's response to therapy, wherein the patient is suffering or suspected of suffering from a relapse, or is at risk of suffering a relapse, the method comprising:
a. providing a sample obtained from the patient, wherein the patient has received therapy for a relapse;
b. comparing an intensity of one or more chemical shift region(s) of a ¹H-NMR spectrum of the sample obtained from the patient with the intensity of the same one or more chemical shift region(s) of a ¹H-NMR reference standard, wherein the one or more chemical shift region(s) are selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, 1.53-1.61 ppm, 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm; and
c. determining that the patient is responsive to the therapy when:
i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is lower in the sample relative to the reference standard, wherein the reference standard is a reference standard that is representative of the intensity of said one or more chemical shift region(s) in a sample obtained from the patient pre-administration of the therapy; and/or
ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is higher in the sample relative to the reference standard, wherein the reference standard is a reference standard that is representative of the intensity of said one or more chemical shift region(s) in a sample obtained from the patient pre-administration of the therapy; or
d. not determining that the patient is responsive to the therapy, or determining that the patient is not responsive to the therapy, when:
i. the intensity of one or more chemical shift region(s) selected from: 1.37-1.55 ppm, 1.65-1.75 ppm, 1.83-1.94 ppm, 3.00-3.05 ppm, 2.80-3.00 ppm, 3.96-4.02 ppm, 3.17-3.95 ppm, 4.63-4.66 ppm, 5.22-5.25 ppm, and 1.53-1.61 ppm is the same or higher in the sample relative to the reference standard, wherein the reference standard is a reference standard that is representative of the intensity of said one or more chemical shift region(s) in a sample obtained from the patient pre-administration of the therapy; and/or
ii. the intensity of one or more chemical shift region(s) selected from: 0.94-0.98 ppm, 1.62-1.78 ppm, 3.70-3.79 ppm, 7.32-7.44 ppm, 3.1-3.3 ppm, 3.9-4.0 ppm, 1.19-1.21 ppm, 2.27-2.45 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, and 1.93-2.10 ppm is the same or lower in the sample relative to the reference standard, wherein the reference standard is a reference standard that is representative of the intensity of said one or more chemical shift region(s) in a sample obtained from the patient pre-administration of the therapy.

7. The method according to claim 1, further comprising:
a. comparing a concentration of isoleucine and/or serum neurofilament light chain (NfL) present in a sample obtained from a patient with the concentration of isoleucine and/or NfL, respectively, in a reference standard; and
b. confirming that the patient is suffering from a relapse when:
i. the concentration of NfL is higher in the sample relative to the reference standard, wherein the reference standard is a non-relapse reference standard; and/or
ii. the concentration of isoleucine is lower in the sample relative to the reference standard, wherein the reference standard is a non-relapse reference standard; and/or
iii. the concentration of NfL is the same or higher in the sample relative to the reference standard, wherein the reference standard is a relapse reference standard; and/or
iv. the concentration of isoleucine is the same or lower in the sample relative to the reference standard, wherein the reference standard is a relapse reference standard; or
c. not confirming that the patient is suffering from a relapse when:
i. the concentration of NfL is the same or lower in the sample relative to the reference standard, wherein the reference standard is a non-relapse reference standard; and/or
ii. the concentration of isoleucine is the same or higher in the sample relative to the reference standard, wherein the reference standard is a non-relapse reference standard; and/or
iii. the concentration of NfL is lower in the sample relative to the reference standard, wherein the reference standard is a relapse reference standard; and/or
iv. the concentration of isoleucine is higher in the sample relative to the reference standard, wherein the reference standard is a relapse reference standard.

8. The method according to claim 1, further comprising:
a. comparing an intensity of a chemical shift region of a ¹H-NMR spectrum of a sample obtained from a patient with the intensity of the same one or more chemical shift region of a ¹H-NMR reference standard, wherein the chemical shift region is 0.92-0.97 ppm, 1.00-1.03 ppm, 1.22-1.28 ppm, 1.43-1.51 ppm, 1.94-2.01 ppm, and/or 3.65-3.68 ppm; and
b. confirming that the patient is suffering from a relapse when:
i. the intensity of said chemical shift region of the ¹H-NMR spectrum is lower in the sample relative to the reference standard, wherein the reference standard is a non-relapse reference standard; and/or
ii. the intensity of said chemical shift region of the ¹H-NMR spectrum is the same or lower in the sample relative to the reference standard, wherein the reference standard is a relapse reference standard; or
c. not confirming that the patient is suffering from a relapse when:
i. the intensity of said chemical shift region of the ¹H-NMR spectrum is the same or higher in the sample relative to the reference standard, wherein the reference standard is a non-relapse reference standard; and/or
ii. the intensity of said chemical shift region of the ¹H-NMR spectrum is higher in the sample relative to the reference standard, wherein the reference standard is a relapse reference standard.

9. The method according to claim 1, further comprising:
a. comparing a concentration of isoleucine and/or serum neurofilament light chain (NfL) present in a sample obtained from a patient with the concentration of isoleucine and/or NfL, respectively, in a reference standard; and
b. determining the patient's prognosis is poor when:
i. the concentration of NfL is higher in the sample relative to the reference standard, wherein the reference standard is a non-relapse reference standard; and/or
ii. the concentration of isoleucine is lower in the sample relative to the reference standard, wherein the reference standard is a non-relapse reference standard; and/or
iii. the concentration of NfL is the same or higher in the sample relative to the reference standard, wherein the reference standard is a relapse reference standard; and/or
iv. the concentration of isoleucine is the same or lower in the sample relative to the reference standard, wherein the reference standard is a relapse reference standard; or
c. not determining the patient's prognosis is poor when:
i. the concentration of NfL is the same or lower in the sample relative to the reference standard, wherein the reference standard is a non-relapse reference standard; and/or
ii. the concentration of isoleucine is the same or higher in the sample relative to the reference standard, wherein the reference standard is a non-relapse reference standard; and/or
iii. the concentration of NfL is lower in the sample relative to the reference standard, wherein the reference standard is a relapse reference standard; and/or
iv. the concentration of isoleucine is higher in the sample relative to the reference standard, wherein the reference standard is a relapse reference standard.

10. The method according to claim 1, further comprising:
a. comparing an intensity of a chemical shift region of a ¹H-NMR spectrum of a sample obtained from a patient with the intensity of the same one or more chemical shift region of a ¹H-NMR reference standard, wherein the chemical shift region is 0.92-0.97 ppm, 1.00-1.03 ppm, 1.22-1.28 ppm, 1.43-1.51 ppm, 1.94-2.01 ppm, and/or 3.65-3.68 ppm; and
b. determining the patient's prognosis is poor when:
i. the intensity of said chemical shift region of the ¹H-NMR spectrum is lower in the sample relative to the reference standard, wherein the reference standard is a non-relapse reference standard; and/or
ii. the intensity of said chemical shift region of the ¹H-NMR spectrum is the same or lower in the sample relative to the reference standard, wherein the reference standard is a relapse reference standard; or
c. not determining the patient's prognosis is poor when:
i. the intensity of said chemical shift region of the ¹H-NMR spectrum is the same or higher in the sample relative to the reference standard, wherein the reference standard is a non-relapse reference standard; and/or
ii. the intensity of said chemical shift region of the ¹H-NMR spectrum is higher in the sample relative to the reference standard, wherein the reference standard is a relapse reference standard.

11. The method according to claim 1, further comprising:
a. comparing a concentration of isoleucine and/or serum neurofilament light chain (NfL) present in a sample obtained from a patient with the concentration of isoleucine and/or NfL, respectively, in a reference standard; and
b. determining that the patient is responsive to the therapy when:
i. the concentration of NfL is lower in the sample relative to the reference standard, wherein the reference standard is a reference standard that is representative of the concentration of said one or more metabolite(s) in a sample obtained from the patient pre-administration of the therapy; or
ii. the concentration of isoleucine is higher in the sample relative to the reference standard, wherein the reference standard is a reference standard that is representative of the concentration of said one or more metabolite(s) in a sample obtained from the patient pre-administration of the therapy; and/or
c. not determining that the patient is responsive to the therapy when:
i. the concentration of NfL is the same or higher in the sample relative to the reference standard, wherein the reference standard is a reference standard that is representative of the concentration of said one or more metabolite(s) in a sample obtained from the patient pre-administration of the therapy; and/or
ii. the concentration of isoleucine is the same or lower in the sample relative to the reference standard, wherein the reference standard is a reference standard that is representative of the concentration of said one or more metabolite(s) in a sample obtained from the patient pre-administration of the therapy.

12. The method according to claim 1, further comprising:
a. comparing an intensity of a chemical shift region of a ¹H-NMR spectrum of a sample obtained from a patient with the intensity of the same one or more chemical shift region of a ¹H-NMR reference standard, wherein the chemical shift region is 0.92-0.97 ppm, 1.00-1.03 ppm, 1.22-1.28 ppm, 1.43-1.51 ppm, 1.94-2.01 ppm, and/or 3.65-3.68 ppm; and
b. determining that the patient is responsive to the therapy when:
i. the intensity of said chemical shift region of the ¹H-NMR spectrum is higher in the sample relative to the reference standard, wherein the reference standard is a reference standard that is representative of the intensity of said one or more chemical shift region(s) in a sample obtained from the patient pre-administration of the therapy; or
c. not determining that the patient is responsive to the therapy when:
i. the intensity of said chemical shift region of the 1H-NMR spectrum is the same or lower in the sample relative to the reference standard, wherein the reference standard is a reference standard that is representative of the intensity of said one or more chemical shift region(s) in a sample obtained from the patient pre-administration of the therapy.

13. (canceled)

14. The method according to claim 1, wherein the sample is a biofluid sample, preferably wherein the sample is a blood sample.

15. (canceled)

16. The method according to claim 1, wherein the concentration of the one or more metabolites has been, or is, determined using ¹H-NMR spectroscopy.

17. The method according to claim 1, wherein the chemical shift region(s) is/are reported relative to lactate —CH₃ referenced at 1.33 ppm.

18. (canceled)

19. The method according to claim 1, wherein the method comprises comparing the concentration two or more metabolites or comparing the intensity of two or more chemical shift regions, respectively.

20. The method according to claim 1, wherein the method comprises comparing the concentration of three or more metabolites or comparing the intensity of three or more chemical shift regions, respectively.

21. The method according to claim 1, wherein the patient has relapsing remitting MS (RRMS).

22. The method according to claim 1 further comprising recording the output of at least one step on a data-storage medium.

23-24. (canceled)

25. A method of treating a relapse in a multiple sclerosis (MS) patient, the method comprising:
a. obtaining the results of a method according to claim 1; and
b. administering a therapy for treating relapse when a relapse is confirmed; and
c. optionally administering a different therapy when a relapse is not confirmed.
